(19) United States
(12) Patent Application Publication
(10) Pub. No.: US 2021/0386678 A1
(43) Pub. Date: Dec. 16, 2021

(54) BIOPOLYMER-ENCAPSULATED GLYCOSYL TRANSFERASE INHIBITOR COMPOSITIONS AND METHODS FOR TREATING DIABETES AND CARDIAC INDICATIONS

(71) Applicant: The Johns Hopkins University, Baltimore, MD (US)

(72) Inventor: Subroto Chatterjee, Columbia, MD (US)

(21) Appl. No.: 17/152,708

(22) Filed: Jan. 19, 2021

Related U.S. Application Data
(63) Continuation of application No. 15/306,813, filed on Oct. 26, 2016, now abandoned.
(60) Provisional application No. 62/126,190, filed on Feb. 27, 2015.
(60) Provisional application No. 61/985,154, filed on Apr. 28, 2014.

(51) Int. Cl. A61K 9/50; A61K 31/5375; A61K 45/06; A61K 9/00

(52) U.S. Cl. CPC A61K 9/5031; A61K 9/0053; A61K 45/06; A61K 31/5375

(57) ABSTRACT

Biopolymer-Encapsulated Glycosyltransferase Inhibitor Compositions and Methods for Treating Diabetes and Cardiac Indications.

### RELATED APPLICATIONS

The present invention claims priority to, and the benefit under 35 U.S.C. § 119(e) of U.S. provisional patent application No. 62/126,190, entitled “Biopolymer-encapsulated glycosyltransferase inhibitor compositions and methods for treating diabetes and cardiac indications,” filed Feb. 27, 2015, and of U.S. provisional patent application No. 61/985,154, entitled, “Biopolymer-encapsulated glycosyltransferase inhibitor compositions and methods for treating cardiac indications,” filed Apr. 28, 2014. The entire contents of the aforementioned patent applications are incorporated herein by this reference.

### FIELD OF THE INVENTION

The present invention relates to compositions and methods for treating or reducing a symptom of diabetes, atherosclerosis, and cardiac hypertrophy.

### BACKGROUND OF THE INVENTION

Atherosclerosis and cardiac hypertrophy contribute to nearly one half of the mortality and morbidity in the Western Hemisphere. Various risk factors contribute to atherosclerosis among which accumulation of cholesterol and triglycerides contribute to plaque formation. Since glycosphingolipids are carried on lipoproteins, they too accumulate in atherosclerotic plaques and blood levels of glycosphingolipids rise when blood cholesterol levels increase. Although lowering blood cholesterol levels using the statins and cholesterol absorption inhibitors has met with great success, very little is known about whether lowering the glycosphingolipid load can affect atherosclerosis or cardiac hypertrophy.

D-threo-1-phenyl-2-decanoylamino-3-morpholino-1-propanol (D-PDMP) is an inhibitor of glycosphingolipid synthesis. Although D-PDMP is well tolerated by experimental animals, it has a very short half-life (approximately 52 minutes) because it is rapidly metabolized. Unfortunately, the poor pharmacological properties of D-PDMP comprise the therapeutic value of D-PDMP as a glycosphingolipid synthesis inhibitor. Therefore, there exists an urgent need for compositions and methods to improve the pharmacological properties of a glycosphingolipid synthesis inhibitor.

### SUMMARY OF THE INVENTION

The present invention provides compositions and methods for treating or reducing a symptom of atherosclerosis or cardiac hypertrophy and/or diabetes. It is contemplated within the scope of the invention that the compositions and methods herein may also be used to treat diseases such as, for example, diabetes, cerebral atherosclerosis, obesity, metabolic syndrome, fibrosis of the lungs and kidney, and renal cancer and tuberculosis, Niemann-Pick Type C (NPC), Alzheimer's, and epilepsy, as well as several metabolic disorders of glycosphingolipid metabolism such as, for example, fibrosis of liver, inflammation (macrophage infiltration into the heart and coronary artery), Gaucher's disease (glucocerebrosidase deficiency) and Fabry's disease (ceramide trihexoside deficiency), in which cardiac hypertrophy has been documented.

In one aspect, the invention generally provides a composition for treating or reducing a symptom of atherosclerosis or cardiac hypertrophy comprising an effective amount of a biopolymer-encapsulated glycosphingolipid synthesis inhibitor. In an embodiment, the glycosphingolipid synthesis inhibitor is D-threo-1-phenyl-2-decanoylamino-3-morpholino-1-propanol (D-PDMP). In an embodiment, the D-PDMP is encapsulated in a polyethylene glycol-sebacic acid polymer. In any of the above embodiments, the composition can further include another therapeutic compound known to treat or reduce a symptom of heart disease generally and atherosclerosis or cardiac hypertrophy specifically. A composition of the present invention can be for oral administration of any drug to escape the acidic pH of the stomach.

In another aspect, the invention generally provides a method for treating or reducing a symptom of atherosclerosis or cardiac hypertrophy in a subject in need thereof comprising a step of providing a composition comprising effective amount of a biopolymer-encapsulated glycosphingolipid synthesis inhibitor to the subject. In an embodiment, the glycosphingolipid synthesis inhibitor is D-threo-1-phenyl-2-decanoylamino-3-morpholino-1-propanol (D-PDMP). In an embodiment the D-PDMP is encapsulated in a polyethylene glycol—sebacic acid polymer. In any of the above embodiments, the method can include providing another therapeutic compound known to treat or reduce a symptom of heart disease generally and atherosclerosis or cardiac hypertrophy specifically. In embodiments, D-PDMP is provided at 0.1 mg to 100 mg per kg of subject bodyweight, e.g., 1 mg or 10 mg/kg. In any of the above embodiments the subject is a mammal, e.g., a human. In any of the above embodiments a composition is orally administered.

In certain embodiments, the composition is a sustained or controlled release dosage formulation. In one embodiment, the cardiac hypertrophy is caused by one or more of the following conditions: atherosclerosis (optionally, cerebral atherosclerosis), obesity, metabolic syndrome, fibrosis of the lungs, fibrosis of the kidney, renal cancer, tuberculosis, Niemann-Pick Type C (NPC), Alzheimer's, epilepsy and/or a metabolic disorder of glycosphingolipid metabolism. Optionally, the metabolic disorder of glycosphingolipid metabolism is fibrosis of liver, inflammation (macrophage infiltration into the heart and coronary artery), Gaucher's disease (glucocerebrosidase deficiency) or Fabry's disease (ceramide trihexo side deficiency).

In another aspect, the invention generally provides a composition and/or method for treating or reducing a symptom of diabetes in a subject including an effective amount of a biopolymer-encapsulated glycosphingolipid synthesis inhibitor.

Another aspect of the invention provides a method for treating or reducing a symptom of Alzheimer's disease (AD) in a subject in need thereof involving providing a composition including an effective amount of a glycosphingolipid synthesis inhibitor to the subject.

Optionally, the glycosphingolipid synthesis inhibitor is biopolymer-encapsulated. In certain embodiments, the glycosphingolipid synthesis inhibitor is D-PDMP.

In some embodiments, the glycosphingolipid synthesis inhibitor is encapsulated in a polyethylene glycol—sebacic acid polymer.

Optionally, amyloid-β levels are reduced and/or amyloid-β plaque levels and/or amyloid-β plaque formation is reduced or eliminated in the treated subject.

Any of the above aspects and embodiments can be combined with any other aspect or embodiment.

Other features and advantages of the invention will be apparent from the detailed description, and from the claims.

### Definitions

Unless defined otherwise, all technical and scientific terms used herein have the meaning commonly understood by a person skilled in the art to which this invention belongs. The following references provide one of skill with a general definition of many of the terms used in this invention: The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them below, unless specified otherwise.

The term “atherosclerosis” refers to a condition in which an artery wall thickens as the result of a build-up of fatty materials such as cholesterol. It is commonly referred to as a hardening or furring of the arteries. It is caused by the formation of multiple plaques within the arteries. It is a syndrome affecting arterial blood vessels, a chronic inflammatory response in the walls of arteries, in large part due to the accumulation of macrophage white blood cells and promoted by oxidized low-density lipoproteins (plasma proteins that carry cholesterol and triglycerides) without adequate removal of fats and cholesterol from the macrophages by functional high density lipoproteins (HDL). Atherosclerosis can affect any artery in the body, including arteries in the heart, brain, arms, legs, pelvis, and kidneys. As a result, different diseases may develop based on which arteries are affected. Atherosclerosis-Related Diseases include but is not limited to, Coronary Heart Disease (CHD), Carotid Artery Disease, Peripheral arterial disease (P.A.D.) and Chronic Kidney Disease.

The term “cardiac hypertrophy” refers to a thickening of the heart muscle (myocardium) which results in a decrease in size of the chamber of the heart, including the left or right ventricles. Cardiac hypertrophy is an adaptive response to pressure or volume stress, mutations of sarcomeric (or other) proteins, or loss of contractile mass from prior infarction. Hypertrophic growth accompanies many forms of heart disease, including ischemic disease, hypertension, heart failure, and valvular disease. In these types of cardiac pathology, pressure overload-induced concentric hypertrophy is believed to have a compensatory function by diminishing wall stress and oxygen consumption.

By “heart disease” is meant related diseases, including, but not limited to: coronary heart disease (CHD), cardiomyopathy, cardiovascular disease (CVD), ischemic heart disease, heart failure, hypertensive heart disease, inflammatory heart disease, valvular heart disease, atherosclerosis, cardiac hypertrophy, fibrosis of liver, and inflammation (macrophage infiltration into the heart and coronary artery. Heart disease is a systemic disease that can affect the heart, brain, most major organs, and the extremities. By “coronary heart disease (CHD)” is meant a disease that causes the failure of coronary circulation to supply adequate circulation to the cardiac muscles and surrounding tissues. By “cardiovascular disease (CVD)” is meant any of a number of specific diseases that affect the heart itself or the blood vessel system, especially the myocardial tissue, as well as veins and arteries leading to and from the heart. For example, CVD may include, but is not limited to, acute coronary syndromes, arrhythmia, atherosclerosis, heart failure, myocardial infarction, neointimal hyperplasia, pulmonary hypertension, stroke, valvular disease, or cardiac hypertrophy. Heart disease may be diagnosed by any of a variety of methods known in the art. For example, such methods may include assessing a subject for dyspnea, orthopnea, paroxysmal nocturnal dyspnea, claudication, angina, chest pain, which may present as any of a number of symptoms known in the art, such as exercise intolerance, edema, palpitations, faintness, loss of consciousness, or cough; heart disease may be diagnosed by blood chemistry analysis. As described above and as used herein, “heart disease” relates to a disorder affecting the heart itself or the circulatory system.

A subject that is “diagnosed with atherosclerosis” or “cardiac hypertrophy” presents with one or more symptoms of atherosclerosis or cardiac hypertrophy known to one of skill in the art as assessed by physical exam, murmur, carotid ultrasound, echocardiography, CT, MRI, stress test ECG, nuclear stress test, stress test with echocardiography, angiography or blood chemistry analysis.

As used herein, atherosclerosis or cardiac hypertrophy is treated or a symptom thereof is reduced if, for example, a patient displays angiographic resolution of an atherosclerotic plaque burden as seen by angiography or IVUS, lowering of blood-borne indicators of atherosclerosis or cardiac hypertrophy, e.g., plasma LDL cholesterol levels, as assessed by blood chemistry analysis, reduction in blood pressure, improvement of exercise tolerance as observed by stress test, or reduction in the mass or size of cardiac musculature.

The term “symptoms” as it refers to atherosclerosis or cardiac hypertrophy are as described herein and as well-known to those skilled in the art. The present invention relates to reducing a symptom of atherosclerosis or cardiac hypertrophy.

The term “diabetes”, as used herein, has its art-recognized meaning and refers to a group of metabolic diseases that affect how the body uses sugar, often resulting in prolonged periods of high blood sugar levels, or hyperglycemia. The two most common types of diabetes are classified as type 1 and type 2. The classic symptoms of untreated diabetes include weight loss, presence of ketones in the urine, fatigue, polyuria, polydipsia, and polyphagia. Symptoms may develop rapidly (weeks or months), or can develop much more slowly or be absent in type 2 diabetes. Over time, high blood glucose levels can damage nerves and blood vessels, leading to complications that may be disabling or life-threatening. Some possible complications include cardiovascular disease (including angina, heart attacks, and atherosclerosis), nerve damage, and kidney damage.

Type I diabetes is a chronic autoimmune disease characterized by the destruction of insulin-producing cells of the pancreas. Destruction of, e.g., β-islet cells of the pancreas leaves the patient with little or no insulin, and instead of being transported into cells, sugar builds up in the bloodstream. It is currently estimated that of the more than 387 million diabetics worldwide, 5-10% have type I diabetes. This form is commonly referred to as “insulin-dependent diabetes mellitus” or “juvenile diabetes,” as it typically appears during childhood or adolescence. The underlying cause is unknown; however, heredity plays an important role in determining a person's likelihood of developing diabetes, as do environmental factors. Although there is no cure for type I diabetes, it can be managed with daily insulin injections.

Type II diabetes begins with insulin resistance, in which cells fail to respond to insulin properly, and continued progression of the disease may result in a lack of insulin production. Type II diabetes is the form that afflicts roughly 90% of diabetic patients worldwide. The primary cause is excessive body weight and not enough exercise. Likewise, prevention and treatment commonly involve instigation of a healthy diet, physical exercise, not using tobacco and obtaining and maintaining a normal body weight. Cells in patients with type 2 diabetes become resistant to insulin, and the pancreas is unable to overcome this resistance. Similar to type I diabetes, sugar builds up in the bloodstream instead of moving into cells where it is utilized for energy. Type 2 diabetes can be treated with medications, with or without insulin. Typically, insulin and some oral medications can cause low blood sugar. In certain instances of

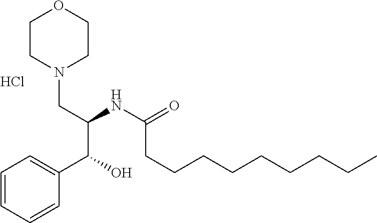 [SMILES: CCCCCCCCCC(=O)N[C@H](CN1CCOCC1)[C@H](O)c1ccccc1 ; Cl]

obese subjects having type 2 diabetes, weight loss surgery can also be an effective treatment. D-threo-1-phenyl-2-decanoylamino-3-morpholino-1-propanol (D-PDMP) is an inhibitor of glycosphingolipid synthesis. It has the following structure:

By “glycosphingolipid” is meant a subtype of glycolipids containing the amino alcohol sphingosine. They may be considered sphingolipids with a carbohydrate attached. Examples of glycosphingolipid include Cerebrosides, Gangliosides, and Globosides.

The term “administering,” as used herein, refers to any mode of transferring, delivering, introducing, or transporting a therapeutic agent to a subject in need of treatment with such an agent. Such modes include, but are not limited to, oral, topical, intravenous, intraperitoneal, intramuscular, intradermal, intranasal, and subcutaneous administration.

By “agent” is meant any small molecule chemical compound, antibody, nucleic acid molecule, or polypeptide, or fragments thereof. An “agent” includes a “therapeutic agent” as defined herein below.

By “alteration” is meant a change (increase or decrease) in the expression levels or activity of a gene or polypeptide as detected by standard art known methods such as those described herein. As used herein, an alteration includes a 10% or more change in expression levels or activity of a gene or polypeptide, preferably a 25% change, more preferably a 40% change, and most preferably a 50% or greater change in expression levels or activity of a gene or polypeptide.

As used herein an “alteration” also includes a 2-fold or more change in expression levels or activity of a gene or polypeptide, for example, 5-fold, 10-fold, 20-fold, 30-fold, 40-fold, 50-fold, 100-fold, 500-fold, 1000-fold or more.

By “ameliorate” is meant decrease, suppress, attenuate, diminish, arrest, or stabilize the development or progression of a disease such as, for example, atherosclerosis or cardiac hypertrophy.

By “amplify” is meant to increase the number of copies of a molecule. In one example, the polymerase chain reaction (PCR) is used to amplify nucleic acids.

By “binding” is meant having a physicochemical affinity for a molecule. Binding is measured by any of the methods of the invention, e.g., a drug/compound with a molecule expressed on a target cell.

By “biological sample” is meant any tissue, cell, fluid, or other material derived from an organism (e.g., human subject).

By “chemical agent” is meant any chemical compound. For example, a chemical agent may be a small molecule chemical compound that inhibits glycosphingolipid synthesis, thereby reducing the risk of atherosclerosis or cardiac hypertrophy.

In this disclosure, “comprises,” “comprising,” “containing,” “having,” and the like can have the meaning ascribed to them in U.S. Patent law and can mean “includes,” “including,” and the like; the terms “consisting essentially of” or “consists essentially” likewise have the meaning ascribed in U.S. Patent law and these terms are open-ended, allowing for the presence of more than that which is recited so long as basic or novel characteristics of that which is recited are not changed by the presence of more than that which is recited, but excludes prior art embodiments.

As used herein, a “controlled release dosage formulation” refers to a formulation of a drug that offers prolonged release at a specific controllable rate.

“Detect” refers to identifying, either directly or indirectly, the presence, absence, or amount of the lipoprotein to be detected.

By “effective amount” is meant the amount required to ameliorate the symptoms of a disease relative to an untreated patient. The effective amount of active compound(s) used to practice the present invention for therapeutic treatment of a disease varies depending upon the manner of administration, the age, body weight, and general health of the subject. Ultimately, the attending physician or veterinarian will decide the appropriate amount and dosage regimen. Such amount is referred to as an “effective” amount.

Ranges provided herein are understood to be shorthand for all of the values within the range. For example, a range of 1 to 50 is understood to include any number, combination of numbers, or sub-range from the group consisting of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50, as well as all intervening decimal values between the aforementioned integers such as, for example, 1.1, 1.2, 1.3, 1.4, 1.5, 1.6, 1.7, 1.8, and 1.9. With respect to sub-ranges, “nested sub-ranges” that extend from either end point of the range are specifically contemplated. For example, a nested sub-range of an exemplary range of 1 to 50 may comprise 1 to 10, 1 to 20, 1 to 30, and 1 to 40 in one direction, or 50 to 40, 50 to 30, 50 to 20, and 50 to 10 in the other direction.

By “reduces” is meant a negative alteration of at least 10%, 25%, 50%, 75%, or 100%.

By “reference” is meant a standard or control condition.

As used herein, a “sustained release dosage formulation” is a formulation of a drug designed to release the drug at a predetermined rate in order to maintain a constant drug concentration for a specific period of time with minimum side effects. Optionally, the period of time is 30 minutes or more, e.g., 2-4 hours or more, e.g., 3-8 hours or more, e.g., 4-24 hours or more, e.g., 1-3 days or more, e.g., 2-7 days or more, e.g., 4-14 days or more, e.g., 7 days or more, e.g., 14 days to a month or more.

As used herein, “control subject” means a subject that has not been diagnosed with a disease according to the invention, for example atherosclerosis or cardiac hypertrophy, or does not exhibit any detectable symptoms associated with these diseases. A “control subject” also means a subject that is not at risk of developing a disease, for example atherosclerosis or cardiac hypertrophy, as defined herein.

Unless defined otherwise, all technical and scientific terms used herein have the meaning commonly understood by a person skilled in the art to which this invention belongs. Unless specifically stated or obvious from context, as used herein, the terms “a,” “an,” and “the” are understood to be singular or plural. Unless specifically stated or obvious from context, as used herein, the term “or” is understood to be inclusive.

Unless specifically stated or obvious from context, as used herein, the term “about” is understood as within a range of normal tolerance in the art, for example within 2 standard deviations of the mean. About can be understood as within 10%, 9%, 8%, 7%, 6%, 5%, 4%, 3%, 2%, 1%, 0.5%, 0.1%, 0.05%, or 0.01% of the stated value. Unless otherwise clear from the context, all numerical values provided herein are modified by the term “about.”

The invention may be more completely understood in consideration of the following detailed description of various embodiments of the invention.

### BRIEF DESCRIPTION OF THE DRAWINGS

FIGS. 1A-1E depict a scintigraph and four line graphs, respectively. FIG. 1A includes scintigraphs demonstrating progress of a radiolabeled biopolymer through an animal. FIGS. 1B to 1E are graphs showing levels of D-PDMP in various tissues at specified times.

FIGS. 2A-2I a series of histological sections and eight bar graphs, respectively. FIG. 2A shows Masson Trichrome-stained thin aortic tissue sections from mice receiving specified treatment protocols. FIG. 2B is a graph showing intima media thickness in the ascending aorta of mice receiving specified treatment protocols. FIGS. 2C to 21 are graphs showing levels of particular molecules in mice receiving specified treatment protocols.

FIGS. 3A-3C depict three bar graphs, respectively. FIGS. 3A and 3B are graphs showing changes in the heart left ventricular mass and in fractional shortening in mice receiving specified treatment protocols. FIG. 3C is a graph showing airway smooth muscle cell proliferation induced by overexpression of GalT V or GalT VI.

FIGS. 4A-4F are bar graphs showing changes in particular mRNA levels in mice receiving specified treatment protocols.

FIG. 5 is a schematic showing cellular activities related to oxidized LDL influx.

FIGS. 6A-6E depict four images and a graph, respectively. BDP-encapsulated D-PDMP treatment ameliorates atherosclerotic plaque buildup and lumen volume in ApoE−/− mice fed a western diet. Masson Trichrome stained ascending aortic rings of ApoE−/− mouse: Control mice fed regular mice chow (FIG. 6A), mice fed high fat, high cholesterol (HFHC) diet consisting of 20% fat and 1.25 cholesterol plus vehicle (Placebo) (FIG. 6B), HFHC+5 mg/kg D-PDMP (FIG. 6C), and HFHC+10 mg/kg D-PDMP (FIG. 6D). Bar=50 Lumen area is significantly reduced (FIG. 6E) due to increased plaque accumulation in placebo mice aorta. Treatment significantly reduced medial thickening, elastin fibers, and plaque accumulation in a dose-dependent manner. A nonparametric one-way ANOVA using the Kruskal-Wallis test and Dunn's multiple comparison post-test were performed. * p≤0.05, ** p≤0.01, *** p≤0.001; n=3-5.

FIGS. 7A-7D depict 4 graphs, respectively. Plasma levels of oxidized LDL, cholesterol, triglycerides, and HDL-c in ApoE−/− mice fed a high fat and high cholesterol diet with and without BDP-encapsulated D-PDMP. Serum levels of oxLDL (FIG. 7A), LDLc (FIG. 7B), triglycerides (FIG. 7C), and HDLc (FIG. 7D) were determined using an immunohistochemical ELISA assay and LDLc triglycerides and HDLc concentrations were taken from microtiter readings following Wako kit assays. Values are means±SEM. * p≤0.05, ** p 0.01, *** p 0.001; n=3.

FIG. 8 presents a pathway diagram showing D-PDMP as working by inhibiting lactosylceramide synthesis.

FIG. 9 shows an exemplary photo of β-amyloid plaques in cross-sectioned AD brain tissue.

FIGS. 10A and 10B show the ELISA-based detection approach employed to quantify ox-LDL levels.

FIG. 11 shows that D-PDMP decreased atherosclerotic biomarkers ox-LDL and GalT-V in treated AD model mice, in a dose-dependent manner.

FIGS. 12A and 12B show that D-PDMP administration was also observed to have decreased amyloid-β in treated ApoE−/− mice, by both Western (FIG. 12A) and ELISA (FIG. 12B), in an apparently dose-dependent manner.

FIG. 13 shows the extent of correlation between levels of amyloid-β in brain and the atherosclerosis biomarker ox-LDL.

### DETAILED DESCRIPTION OF THE INVENTION

Atherosclerotic heart disease is the main cause of heart attacks and strokes, and is also the number one cause of death among humans. This is due, in part, to high levels of low density lipoprotein (LDL) cholesterol (i.e., bad cholesterol) and triglycerides and low levels of high density lipoprotein (HDL) cholesterol (i.e., good cholesterol), which is collectively referred to as “Metabolic syndrome”. Metabolic syndrome is a major cause of morbidity and mortality, particularly in Western countries and in patients with diabetes. There is a large unmet need to develop drugs to ameliorate this syndrome.

Existing cholesterol-lowering medications approach the problem on a single front, either by blocking cholesterol synthesis (e.g., with statins) or by preventing the body from absorbing too much dietary cholesterol (e.g., with ezetimibe). Unfortunately, these drugs are associated with significant toxicities and are not always effective. For example, statins and ezetimibe may cause myalgias, muscle pain, and gastro-intestinal issues. In addition, statin treatment may cause issues with glucose homeostasis in diabetic patients. For example, an analysis of 13 studies published in the journal Lancet in February 2010 found a 9 percent increased risk of diabetes in people who used statins, which means that there would be one extra case of diabetes for every 255 people who took a statin for four years. Accordingly, diabetics are at a higher risk for developing heart disease, and it is often more severe than in non-diabetics.

According to the techniques herein, abnormal cholesterol production, transport and breakdown can be blocked, thereby successfully preventing the development of atherosclerosis. In particular, the present disclosure shows that it is possible to halt the action of a fat-and-sugar molecule called glycosphingolipid (GSL), which resides in the membranes of all cells and is mostly known for regulating cell growth. GSL also regulates the way the body handles cholesterol. Moreover, as discussed further below, D-threo-1-phenyl-2-decanoylamino-3-morpholino-1-propanol (D-PDMP) blocks the synthesis of GSL, thereby preventing the development of heart disease in mice and rabbits fed a high-fat, cholesterol-laden diet. Without being bound by theory, D-PDMP appears to work by interfering with a constellation of genetic pathways that regulate fat metabolism on multiple fronts: from the way cells derive and absorb cholesterol from food to the way cholesterol is transported to tissues and organs and is then broken down by the liver and excreted. D-PDMP is well tolerated in animals; however, it has a short half-life. According to the techniques herein, D-PDMP may be encapsulated within a Biopolymer (BPD) that releases the D-PDMP in a controlled manner over several days to treat atherosclerotic heart disease.

The invention features compositions and methods for treating and reducing a symptom of atherosclerosis or cardiac hypertrophy or diabetes. The present invention is based, at least in part, on the discovery that a method of providing an animal with an inhibitor of glycosphingolipid synthesis encapsulated in a biopolymer helps reduce unhealthful consequences of a high fat, high cholesterol diet. More specifically, this method is effective in vivo in treating, alleviating, or reducing a symptom associated with atherosclerosis or cardiac hypertrophy such as, for example, fibrosis of liver, and inflammation (macrophage infiltration into the heart and coronary artery. In particular, the compositions and methods of the invention may ameliorate and/or prevent atherosclerosis, as well as other symptoms such as, for example, poor wound healing (e.g., inability or delay of wound healing), that are associated with Type II diabetes. It is contemplated within the scope of the invention that it may also be used to treat diseases such as, for example, diabetes, cerebral atherosclerosis, obesity, metabolic syndrome, fibrosis of the lungs and kidney, Niemann-Pick Type C (NPC), Alzheimer's, and epilepsy, as well as renal cancer and tuberculosis, as well as several metabolic disorders of glycosphingolipid metabolism such as, for example, fibrosis of liver, inflammation (macrophage infiltration into the heart and coronary artery), Gaucher's disease (glucocerebrosidase deficiency) and Fabry's disease (ceramide trihexoside deficiency), in which cardiac hypertrophy has been documented.

According to the techniques herein, encapsulating D-PDMP within a biopolymer composed of polyethylene glycols and sebacic acid not only increases the residence time of the D-PDMP in an animal, but results in an approximately 10-fold increase in efficacy of treatment with the biopolymer-encapsulated D-PDMP when compared to treatments with a non-encapsulated D-PDMP in interfering with atherosclerosis and cardiac hypertrophy in apoE−/− mice fed a high cholesterol, high fat diet.

The therapeutic methods of the invention (which include prophylactic treatment) in general comprise administration of a therapeutically effective amount of the compounds herein, such as a biopolymer encapsulated D-PDMP to a subject in need thereof, including a mammal, particularly a human. Such treatment will be suitably administered to subjects, particularly humans, suffering from, having, susceptible to, or at risk for a heart disease generally and atherosclerosis or cardiac hypertrophy specifically, as well as diabetes. Determination of those subjects “at risk” can be made by any objective or subjective determination by a diagnostic test or opinion of a subject or health care provider (e.g., genetic test, enzyme or protein marker, marker (as defined herein), family history, as well as other medically-accepted indicators).

In various embodiments, the method can include co-treatment of a composition of the present invention along with one or more other therapeutic compound known to treat or reduce a symptom of heart disease generally and atherosclerosis or cardiac hypertrophy specifically. For example, co-treatment can occur with one or more drugs, including Angiotensin converting enzyme (ACE) inhibitors including Capoten® (captopril), Vasotec® (enalapril), Prinivil®, Zestril® (lisinopril), Lotensin® (benazepril), Monopril® (fosinopril), Altace® (ramipril), Accupril® (quinapril), Aceon® (perindopril), Mavik® (trandolapril), and Univasc® (moexipril)); Angiotensin II receptor blockers (ARBs) including Cozaar® (losartan), Diovan® (valsartan), Avapro® (irbesartan), Atacand® (candesartan), and Micardis® (telmisartan); Antiarrhythmia drugs including Tambocor® (flecainide), Procanbid® (procainamide), Cordarone® (amiodarone), and Betapace® (sotalol); Antiplatelet drugs; Beta Blockers including Sectral® (acebutolol), Zebeta® (bisoprolol), Brevibloc® (esmolol), Inderal® (propranolol), Tenormin® (atenolol), Normodyne®, Trandate® (labetalol), Coreg® (carvedilol), Lopressor®, and Toprol-XL® (metoprolol); and Calcium Channel Blockers including Norvasc® (amlodipine), Plendil® (felodipine), Cardizem®, Cardizem CD®, Cardizem SR®, Dilacor XR®, Diltia XT®, Tiazac® (diltiazem), Calan®, Calan SR®, Covera-HS®, Isoptin®, Isoptin SR®, Verelan®, Verelan PM® (verapamil), Adalat®, Adalat CC®, Procardia®, Procardia XL® (nifedipine), Cardene®, Cardene SR® (nicardipine), Sular® (nisoldipine), Vascor® (bepridil); aspirin; digoxin; diuretic drugs; Heart Failure Drugs including Dobutrex® (dobutamine) and Primacor® (milrinone); Vasodialators such as Dilatrate-SR®, Iso-Bid®, Isonate®, Isorbid®, Isordil®, Isotrate®, Sorbitrate® (isosorbide dinitrate), IMDUR® (isorbide mononitrate), Apresoline® (hydralazine), and BiDil® (hydralazine with isosorbide dinitrate); and warfarin. In one preferred embodiment, an agent of the invention is administered in combination with a statin, such as Advicor® (niacin extended-release/lovastatin), Altoprev® (lovastatin extended-release), Caduet® (amlodipine and atorvastatin), Crestor® (rosuvastatin), Lescol® (fluvastatin), Lescol XL (fluvastatin extended-release), Lipitor® (atorvastatin), Mevacor® (lovastatin), Pravachol® (pravastatin), Simcor® (niacin extended-release/simvastatin), Vytorin® (ezetimibe/simvastatin), and Zocor® (simvastatin).

Moreover, the method can be combined with any other method for treating or reducing a symptom of heart disease generally and atherosclerosis or cardiac hypertrophy specifically, as well as diabetes. For example, a surgical procedure or a change in a subject's lifestyle, i.e., diet or exercise. The following examples are put forth so as to provide those of ordinary skill in the art with a complete disclosure and description of how to make and use the assay, screening, and therapeutic methods of the invention, and are not intended to limit the scope of the invention.

The invention also contemplates any derivative form of the aforementioned to pharmaceutical agents and/or therapeutic compounds. Common derivatizations may include, for example, adding a chemical moiety to reduce toxicity, improve solubility and/or stability, and the like, or adding a targeting moiety, which allows more specific targeting of the molecule to a specific cell or region of the body. The pharmaceutical agents and/or therapeutic compounds may also be formulated in any suitable combination, wherein the drugs may either mixed in individual form or coupled together in a manner that retains the functionality of each drug. In addition, the drugs, or a portion thereof, may be modified with fluorescence compound or other detectable labels which may allow tracking of the drug or agent in the body to assess localization, release kinetics, etc.

### Pharmaceutical Compositions

The invention provides pharmaceutical compositions for use in any of the methods described herein. The pharmaceutical compositions may contain a pharmaceutical agents and/or therapeutic compounds.

The pharmaceutical compositions may include a pharmaceutically acceptable carrier. The term “pharmaceutically acceptable” means approved by a regulatory agency of the Federal or a state government or listed in the U.S. Pharmacopeia or other generally recognized pharmacopeia for use in animals, and more particularly in humans. The term “carrier” refers to a diluent, adjuvant, excipient, or vehicle with which the therapeutic is administered. Such pharmaceutical carriers can be sterile liquids, such as water and oils, including those of petroleum, animal, vegetable or synthetic origin, such as peanut oil, soybean oil, mineral oil, sesame oil, olive oil, gel (e.g., hydrogel), and the like. Saline is a preferred carrier when the pharmaceutical composition is administered intravenously. Saline solutions and aqueous dextrose and glycerol solutions can also be employed as liquid carriers, particularly for injectable solutions.

Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol, and the like. The composition, if desired, can also contain minor amounts of wetting or emulsifying agents, or pH buffering agents. These compositions can take the form of solutions, suspensions, emulsion, tablets, pills, capsules, powders, sustained-release formulations and the like. Oral formulation can include standard carriers such as pharmaceutical grades of mannitol, lactose, starch, magnesium stearate, sodium saccharine, cellulose, magnesium carbonate, etc. Examples of suitable pharmaceutical carriers are described in “Remington's Pharmaceutical Sciences” by E. W. Martin, the contents of which are hereby incorporated by reference in its entirety. Such compositions will generally contain a therapeutically effective amount of the pharmaceutical agents and/or therapeutic compounds (e.g., biopolymer encapsulated D-PDMP), in purified form, together with a suitable amount of carrier so as to provide the form for proper administration to the patient. The formulation should suit the mode of administration.

In embodiments, the pharmaceutical agents and/or therapeutic compounds are administered locally as an immediate release or controlled release composition, for example by controlled dissolution and/or the diffusion of the active substance. Dissolution or diffusion controlled release can be achieved by incorporating the active substance into an appropriate matrix. A controlled release matrix may include one or more of a biopolymer, shellac, beeswax, glycowax, castor wax, carnauba wax, stearyl alcohol, glyceryl monostearate, glyceryl distearate, glycerol palmitostearate, ethylcellulose, acrylic resins, dl-polylactic acid, cellulose acetate butyrate, polyvinyl chloride, polyvinyl acetate, vinyl pyrrolidone, polyethylene, polymethacrylate, methylmethacrylate, 2-hydroxymethacrylate, methacrylate hydrogels, 1,3 butylene glycol, ethylene glycol methacrylate, and/or polyethylene glycols and/or sebacic acid. In a controlled release matrix formulation, the matrix material may also include, e.g., hydrated metylcellulose, carnauba wax and stearyl alcohol, carbopol 934, silicone, glyceryl tristearate, methyl acrylate-methyl methacrylate, polyvinyl chloride, polyethylene, and/or halogenated fluorocarbon.

The controlled release matrix may also be a hydrogel: a three-dimensional, hydrophilic or amphiphilic polymeric network capable of taking up large quantities of water. The networks may be composed of homopolymers or copolymers, which are insoluble due to the presence of covalent chemical or physical (e.g., ionic, hydrophobic interactions, entanglements) crosslinks. The crosslinks provide the network structure and physical integrity. Hydrogels exhibit a thermodynamic compatibility with water that allows them to swell in aqueous media. The chains of the network are connected in such a fashion that pores exist and that a substantial fraction of these pores are of dimensions between 1 nm and 1000 nm.

The hydrogels can be prepared by crosslinking hydrophilic biopolymers or synthetic polymers. Examples of the hydrogels formed from physical or chemical crosslinking of hydrophilic biopolymers, include but are not limited to, hyaluronans, chitosans, alginates, collagen, dextran, pectin, carrageenan, polylysine, gelatin, agarose, (meth)acrylate-oligolactide-PEO-oligolactide-(meth)acrylate, poly(ethylene glycol) (PEO), poly(propylene glycol) (PPO), PEO-PPO-PEO copolymers (Pluronics), poly(phosphazene), poly(methacrylates), poly(N-vinylpyrrolidone), PL(G)A-PEO-PL(G)A copolymers, poly(ethylene imine), and the like. See Hennink and van Nostrum, Adv. Drug Del. Rev. 54:13-36 (2002); Hoffman, Adv. Drug Del. Rev. 43:3-12 (2002); Cadee et al., J Control. Release 78:1-13 (2002); Surini et al., J. Control. Release 90:291-301 (2003); and U.S. Pat. No. 7,968,085, each of which is incorporated by reference in its entirety. These materials consist of high-molecular weight backbone chains made of linear or branched polysaccharides or polypeptides.

The amount of the pharmaceutical composition of the invention which will be effective in the treatment or prevention of atherosclerotic heart disease can be determined by standard clinical techniques. In addition, in vitro assays may optionally be employed to help identify optimal dosage ranges. The precise dose to be employed in the formulation may also depend on the route of administration, and the seriousness of the disease, and should be decided according to the judgment of the practitioner and each patient's circumstances. Effective doses may be extrapolated from dose-response curves derived from the in vitro or animal model test systems described herein or known to one of skill in the art.

### Dosages and Administration Regimens

The pharmaceutical agents and/or therapeutic compounds or compositions containing these agents/compounds may be administered in a manner compatible with the dosage formulation, and in such amount as may be therapeutically affective, protective and immunogenic.

The agents and/or compositions may be administered through different routes, including, but not limited to, oral, oral gavage, parenteral, buccal and sublingual, rectal, aerosol, nasal, intramuscular, subcutaneous, intradermal, intraosseous, and topical. The term parenteral as used herein includes, for example, intraocular, subcutaneous, intraperitoneal, intracutaneous, intravenous, intramuscular, intraarticular, intraarterial, intrasynovial, intrastemal, intrathecal, intralesional, and intracranial injection, or other infusion techniques.

In embodiments, the pharmaceutical agents and/or therapeutic compounds formulated according to the present invention are formulated and delivered in a manner to evoke a systemic response. Thus, in embodiments, the formulations are prepared by uniformly and intimately bringing into association the active ingredient with liquid carriers. Formulations suitable for administration include aqueous and non-aqueous sterile solutions, which may contain anti-oxidants, buffers, bacteriostats and solutes which render the formulation isotonic with the blood of the intended recipient, and aqueous and non-aqueous sterile suspensions which may include suspending agents and thickening agents. The formulations may be presented in unit-dose or multi-dose containers, for example, sealed ampoules and vials, and may be stored in a freeze-dried (lyophilized) condition requiring only the addition of the sterile liquid carrier, for example, water, immediately prior to use. Extemporaneous solutions and suspensions may be prepared from sterile powders, granules and tablets commonly used by one of ordinary skill in the art.

The agents and/or compositions may be administered in different forms, including, but not limited to, solutions, emulsions and suspensions, microspheres, particles, microparticles, nanoparticles, liposomes, and the like.

The pharmaceutical agents and/or therapeutic compounds may be administered in a manner compatible with the dosage formulation, and in such amount as may be therapeutically effective, immunogenic and protective. The quantity to be administered depends on the subject to be treated, including, for example, the stage of the disease. Precise amounts of active ingredients required to be administered depend on the judgment of the practitioner. However, suitable dosage ranges are readily determinable by one skilled in the art and may be of the order of micrograms to milligrams of the active ingredient(s) per dose. The dosage may also depend on the route of administration and may vary according to the size of the host.

The pharmaceutical agents and/or therapeutic compounds should be administered to a subject in an amount effective to ameliorate, treat, and/or prevent the disease. Specific dosage and treatment regimens for any particular subject may depend upon a variety of factors, including the activity of the specific compound employed, the age, body weight, general health status, sex, diet, time of administration, rate of excretion, drug combination, the severity and course of the disease (including tumor size), condition or symptoms, the subject's disposition to the disease, condition or symptoms, method of administration, and the judgment of the treating physician. Actual dosages can be readily determined by one of ordinary skill in the art.

Exemplary unit dosage formulations are those containing a dose or unit, or an appropriate fraction thereof, of the administered ingredient. It should be understood that in addition to the ingredients mentioned herein, the formulations of the present invention may include other agents commonly used by one of ordinary skill in the art.

Typically in conventional systemically administered treatments, a therapeutically effective dosage should produce a serum concentration of compound of from about 0.1 ng/ml to about 50-100 μg/ml. The pharmaceutical compositions typically provide a dosage of from about 0.001 mg to about 2000 mg of compound per kilogram of body weight per day. For example, dosages for systemic administration to a human patient can range from 1-10 μg/kg, 20-80 μg/kg, 5-50 μg/kg, 75-150 μg/kg, 100-500 μg/kg, 250-750 μg/kg, 500-1000 μg/kg, 1-10 mg/kg, 5-50 mg/kg, 25-75 mg/kg, 50-100 mg/kg, 100-250 mg/kg, 50-100 mg/kg, 250-500 mg/kg, 500-750 mg/kg, 750-1000 mg/kg, 1000-1500 mg/kg, 1500-2000 mg/kg, 5 mg/kg, 20 mg/kg, 50 mg/kg, 100 mg/kg, 500 mg/kg, 1000 mg/kg, 1500 mg/kg, or 2000 mg/kg. In an exemplary embodiment, an oral dosage for a human weighing 200 kg would be about 200 mg/day. Pharmaceutical dosage unit forms are prepared to provide from about 1 mg to about 5000 mg, for example from about 100 to about 2500 mg of the compound or a combination of essential ingredients per dosage unit form.

In general, a therapeutically effective amount of the present compounds in dosage form usually ranges from slightly less than about 0.025 mg/kg/day to about 2.5 g/kg/day, preferably about 0.1 mg/kg/day to about 100 mg/kg/day of the patient or considerably more, depending upon the compound used, the condition or infection treated and the route of administration, although exceptions to this dosage range may be contemplated by the present invention. It is to be understood that the present invention has application for both human and veterinary use.

The agents and/or compositions are administered in one or more doses as required to achieve the desired effect. Thus, the agents and/or compositions may be administered in 1, 2, to 3, 4, 5, or more doses. Further, the doses may be separated by any period of time, for example hours, days, weeks, months, and years.

The agents and/or compositions can be formulated as liquids or dry powders, or in the form of microspheres.

The agents and/or compositions may be stored at temperatures of from about ˜100° C. to about 25° C. depending on the duration of storage. The agents and/or compositions may also be stored in a lyophilized state at different temperatures including room temperature. The agents and/or compositions may be sterilized through conventional means known to one of ordinary skill in the art. Such means include, but are not limited to, filtration. The composition may also be combined with other anti-atherosclerotic therapeutic agents.

The amount of active ingredient that may be combined with carrier materials to produce a single dosage form may vary depending upon the host treated and the particular mode of administration. In embodiments, a preparation may contain from about 0.1% to about 95% active compound (w/w), from about 20% to about 80% active compound, or from any percentage therebetween.

In embodiments, the pH of the formulation may be adjusted with pharmaceutically acceptable acids, bases, or buffers to enhance the stability of the formulated compound or its delivery form.

In embodiments, the pharmaceutical carriers may be in the form of a sterile liquid preparation, for example, as a sterile aqueous or oleaginous suspension.

Among the acceptable vehicles and solvents that may be employed are mannitol, water, Ringer's solution and isotonic sodium chloride solution.

In addition, sterile, fixed oils are conventionally employed as a solvent or suspending medium. For this purpose, any bland fixed oil may be employed including synthetic mono- or to diglycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant, or carboxymethyl cellulose or similar dispersing agents which are commonly used in the formulation of pharmaceutically acceptable dosage forms such as emulsions and or suspensions.

Other commonly used surfactants such as TWEEN® or SPAN® and/or other similar emulsifying agents or bioavailability enhancers which are commonly used in the manufacture of pharmaceutically acceptable solid, liquid, or other dosage forms may also be used for the purposes of formulation.

In embodiments, the agents and/or compositions can be delivered in an exosomal delivery system. Exosomes are small membrane vesicles that are released into the extracellular environment during fusion of multivesicular bodies with plasma membrane. Exosomes are secreted by various cell types including hematopoietic cells, normal epithelial cells and even some tumor cells.

Also contemplated by the invention is delivery of the pharmaceutical agents and/or therapeutic compounds using nanoparticles. For example, the agents and/or compositions provided herein can contain nanoparticles having at least one or more agents linked thereto, e.g., linked to the surface of the nanoparticle. A composition typically includes many nanoparticles with each nanoparticle having at least one or more agents linked thereto. Nanoparticles can be colloidal metals. A colloidal metal includes any water-insoluble metal particle or metallic compound dispersed in liquid water. Typically, a colloid metal is a suspension of metal particles in aqueous solution. Any metal that can be made in colloidal form can be used, including gold, silver, copper, nickel, aluminum, zinc, calcium, platinum, palladium, and iron. In some cases, gold nanoparticles are used, e.g., prepared from HAuCl4. Nanoparticles can be any shape and can range in size from about 1 nm to about 10 nm in size, e.g., about 2 nm to about 8 nm, about 4 to about 6 nm, or about 5 nm in size. Methods for making colloidal metal nanoparticles, including gold colloidal nanoparticles from HAuCl4, are known to those having ordinary skill in the art. For example, the methods described herein as well as those described elsewhere (e.g., US Pat. Publication Nos. 2001/005581; 2003/0118657; and 2003/0053983, which are hereby incorporated by reference) are useful guidance to make nanoparticles.

In certain cases, a nanoparticle can have two, three, four, five, six, or more active agents linked to its surface. Typically, many molecules of active agents are linked to the surface of the nanoparticle at many locations. Accordingly, when a nanoparticle is described as having, for example, two active agents linked to it, the nanoparticle has two active agents, each having its own unique molecular structure, linked to its surface. In some cases, one molecule of an active agent can be linked to the nanoparticle via a single attachment site or via multiple attachment sites.

An active agent can be linked directly or indirectly to a nanoparticle surface. For example, the active agent can be linked directly to the surface of a nanoparticle or indirectly through an intervening linker.

Any type of molecule can be used as a linker. For example, a linker can be an aliphatic chain including at least two carbon atoms (e.g., 3, 4, 5, 6, 7, 8, 9, 10 or more carbon atoms), and can be substituted with one or more functional groups including ketone, ether, ester, amide, alcohol, amine, urea, thiourea, sulfoxide, sulfone, sulfonamide, and disulfide to functionalities. In cases where the nanoparticle includes gold, a linker can be any thiol-containing molecule. Reaction of a thiol group with the gold results in a covalent sulfide (—S—) bond. Linker design and synthesis are well known in the art.

In embodiments, the nanoparticle is linked to a targeting agent/moiety. A targeting functionality can allow nanoparticles to accumulate at the target at higher concentrations than in other tissues. In general, a targeting molecule can be one member of a binding pair that exhibits affinity and specificity for a second member of a binding pair. For example, an antibody or antibody fragment therapeutic agent can target a nanoparticle to a particular region or molecule of the body (e.g., the region or molecule for which the antibody is specific) while also performing a therapeutic function. In some cases, a receptor or receptor fragment can target a nanoparticle to a particular region of the body, e.g., the location of its binding pair member.

Other therapeutic agents such as small molecules can similarly target a nanoparticle to a receptor, protein, or other binding site having affinity for the therapeutic agent.

When the compositions of this invention comprise one or more additional therapeutic or prophylactic agents, the therapeutic/enhancing/immunotherapy agent and the additional agent should be present at dosage levels of between about 0.1 to 100%, or between about 5 to 95% of the dosage normally administered in a monotherapy regimen. The additional agents may be administered separately, as part of a multiple dose regimen, from the agents of this invention. Alternatively, those additional agents may be part of a single dosage form, mixed together with the agents of this invention in a single composition.

The administration of the pharmaceutical agents and/or therapeutic compounds of the invention elicits, for example, an anti-atherosclerosis response. Typically, the dose can be adjusted within this range based on, e.g., the subject's age, the subject's health and physical condition, the capacity of the subject's immune system to produce an immune response, the subject's body weight, the subject's sex, diet, time of administration, the degree of protection desired, and other clinical factors. Those in the art can also readily address parameters such as biological half-life, bioavailability, route of administration, and toxicity when formulating the agents and/or compositions of the invention.

### EXAMPLES

### Example 1: Biopolymer Encapsulation Increases Controlled Release of D-PDMP

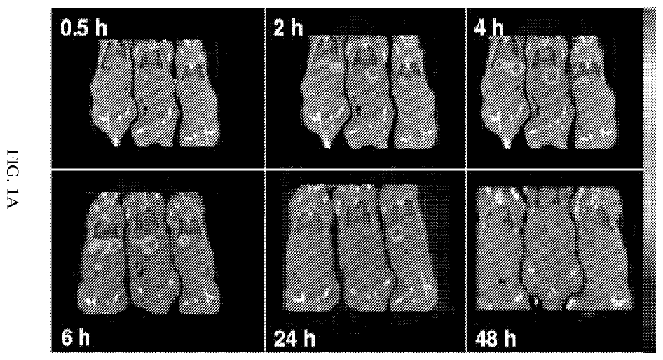

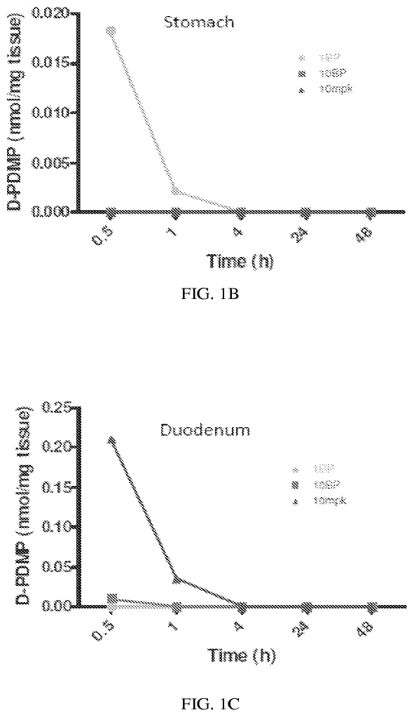

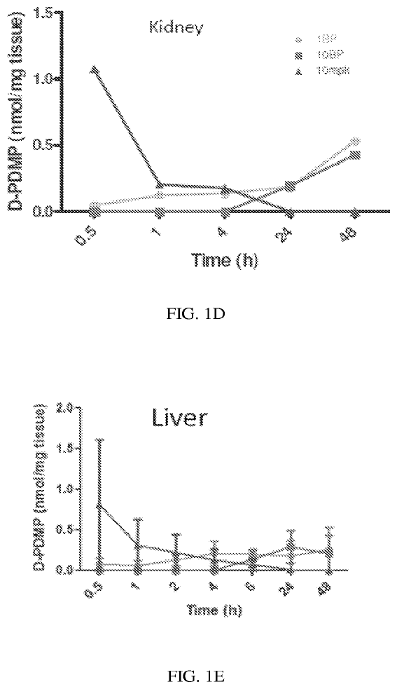

Gamma-irradiation scintigraphy is an established method to image various biomolecules and follow metabolic profiles in experimental animals. This technology was used to image biopolymer encapsulated D-PDMP in C57BL/6 mice using Single-photon emission computed tomography (SPECT). The bio-distribution kinetics of the PEG300 constituent formulated with sebacic acid was investigated by terminally conjugating the PEG with [¹²⁵I]tyrosine. For this, a biopolymer of polyethylene glycol and sebacic acid was radiolabeled with ¹²⁵I. Next, D-PDMP was encapsulated within the biopolymer. Mice were gavaged with the encapsulated D-PDMP and then SPECT imaged. Recordings were made at time points from 0.5 h through 48 h post-gavage and were recorded as indicated in FIG. 1A. Radiotracer uptake, representing the path of the radiolabeled biopolymer, is entirely biliary and is largely dispersed by 24 hours post-administration in two of three mice shown and completely dispersed by 48 hours post-administration. These data support the rapid absorption of D-PDMP in the stomach and duodenum. In contrast, the PEG carrier appears to be absorbed more slowly and accumulates in bile (FIG. 1A) within the gall bladder, suggesting D-PDMP may be liberated from the carrier during GI absorption.

In other experiments, mice were gavaged either with 1 mg/kg D-PDMP encapsulated in biopolymer (1BP), with 10 mg/kg D-PDMP encapsulated in biopolymer (10BP), or with 10 mg/kg non-encapsulated D-PDMP (10 mpk). Mice were imaged (as described above) and then euthanized. Blood was drawn from the euthanized mice and serum was prepared from the blood. Tissues were excised and total lipids were extracted from the excised tissues. Lipid extracts were subjected to mass spectrometric (MS) analysis to determine D-PDMP levels.

To determine the tissue bio-distribution kinetics of both the PEG vehicle and the D-PDMP drug payload in normal mice following oral gavage administration. D-PDMP bio-distributions were determined and compared as encapsulated in the PEG₃₀₀+sebacic acid vehicle versus when administered as free drug. Unlabeled D-PDMP was administered to female C57BL/6 mice either as 10 mpk of free D-PDMP or a biopolymer-encapsulated dose of 1 or 10 mpk mice were then sacrificed at 0.5, 1.0, 2.0, 4.0, 6.0, 24 and 48 h post-gavage. To determine distribution of D-PDMP, whole blood was collected and serum was prepared to quantitate tissue distribution of delivered D-PDMP drug. Various tissues were excised. Total lipids were extracted and subjected to mass spectrometric analysis to determine the levels of total D-PDMP.

The above-described scintigraphic analyses suggest that biopolymer-encapsulated D-PDMP passed through the stomach and duodenum and entered the kidneys in 24 hours (see, FIG. 1A). Thereafter, no radioactivity was detected in the mouse tissues. Since polyethylene glycol is a laxative, it was not appreciably absorbed by the gut; however, it was metabolized in the kidney and excreted.

Quantitative measurement by tandem mass spectrometry of the unconjugated D-PDMP level in the stomach, duodenum and kidneys revealed that within 30 minutes of gavage, a majority of non-encapsulated D-PDMP was absorbed by the stomach and duodenum and was mostly shed in the kidney (see FIGS. 1B to 1E). Thereafter, there was a slow but steady presence of non-encapsulated D-PDMP in these tissues (mostly in kidneys) for one to two hours. Much of the non-encapsulated D-PDMP was eliminated approximately 4 hours after gavage. Very little non-encapsulated D-PDMP was found in these tissues 24 hours after gavage.

In sharp contrast, very little D-PDMP derived from either 1 mg/kg D-PDMP encapsulated in biopolymer or 10 mg/kg D-PDMP encapsulated in biopolymer was found to be associated with the stomach, duodenum or kidneys several hours after administration to the mice by gavage (see FIGS. 1B to 1E). At 24 hours post-gavage and afterwards, a marked increase in D-PDMP associated with the biopolymer was found in the kidneys. Biopolymer-encapsulated D-PDMP was present in the kidneys up to 48 hours afterwards.

Thus, mass spectrometric analysis of mouse tissues showed that non-encapsulated D-PDMP was rapidly metabolized in and excreted by the kidney within an hour of gavage whereas encapsulated D-PDMP was rapidly (<30 min) absorbed in murine GI tissues and displayed a steady and increasing renal excretion profile through 48 h post-gavage. Instead, encapsulated D-PDMP was metabolized in the kidney and excreted.

Together, these data indicated that biopolymer encapsulation increased the residence time of D-PDMP in a mouse's body.

### Example 2: Treatments with a Biopolymer Encapsulated D-PDMP were More Effective in Reducing Aortic Intima Media Thickening in Mice Fed a High Fat and High Cholesterol (HFHC) Diet, as Compared to Treatments with Non-Encapsulated D-PDMP

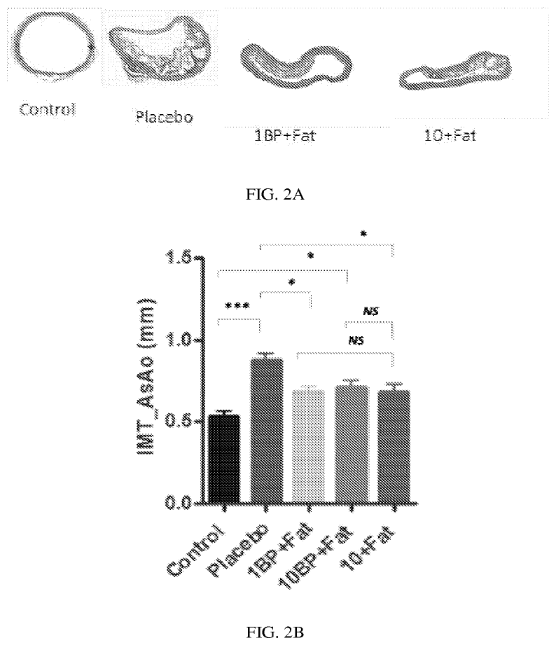

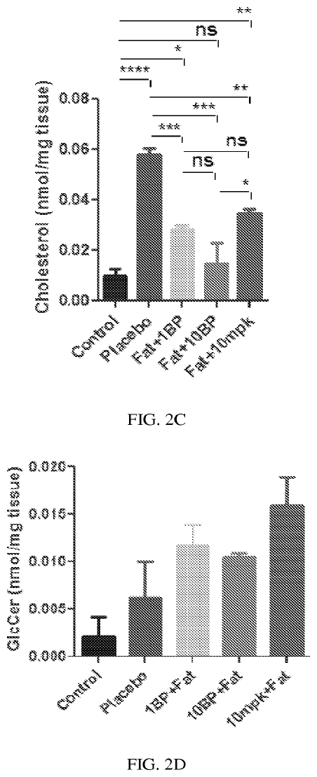

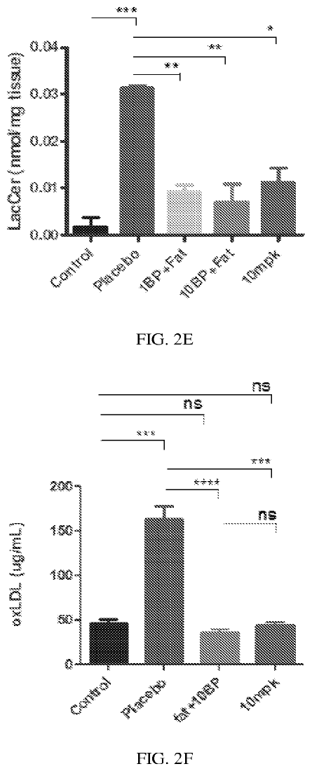

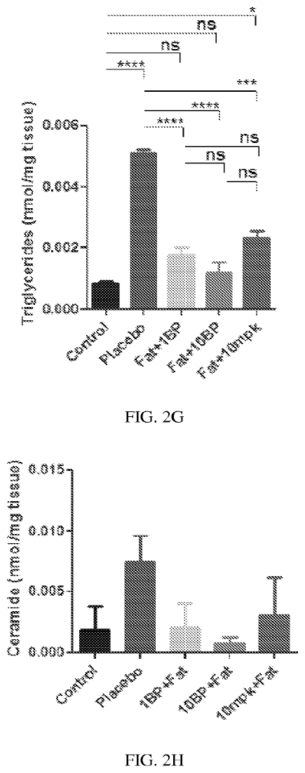

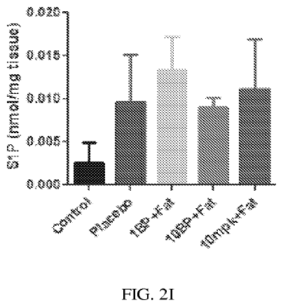

Examination of Masson Trichrome-stained thin aortic tissue sections revealed extensive atherosclerosis plaque buildup, cholesterol ester crystal deposits, calcium accumulation, defragmented elastin fibers, and extensive fibrosis in apolipoprotein E (apoE) −/− mice fed a high fat and high cholesterol (HFHC) diet (FIG. 2A, Placebo). These adverse consequences were markedly interfered/reduced in apoE −/− mice fed a HFHC diet when treated with 1 mg/kg D-PDMP encapsulated in biopolymer (FIG. 2A, 1BP+Fat) from week 24 to week 36. In FIG. 2A, Control mice were apoE −/− mice fed a normal mouse diet and 10+Fat mice are apoE −/− mice fed a HFHC diet and treated with 10 mg/kg non-encapsulated D-PDMP.

FIG. 2B shows graphical representation of intima media thickness in the ascending aorta (IMT-AsAo) obtained using 2D-mode ultrasound imaging. Biopolymer-encapsulated D-DPMP was more effective in interfering/ameliorating IMT-AsAo in apoE −/− mice compared to non-encapsulated D-PDMP.

As shown in FIG. 2B, the unconjugated and encapsulated forms of D-PDMP were equally efficacious in returning the aortic intima-media thickness of the ascending aorta to control levels. This was further examined by measuring the levels of various lipids and lipoproteins in the serum and in liver tissue in these experimental animals (FIGS. 2C to 2F).

Measurement of oxidized LDL (oxLDL) levels using enzyme-linked immunosorbent assay (ELISA) revealed a marked increase in placebo mice serum (FIG. 2E). In sharp contrast, treatment with 1 mg/kg D-PDMP encapsulated in biopolymer (1BP+Fat) interfered/reduced the oxLDL levels to nearly baseline levels. Similarly, mass spectrometry of cholesterol levels or triglyceride levels showed that treatment with 1 mg/kg D-PDMP encapsulated in biopolymer interfered with the rise in the level of these lipids in liver tissue as compared to placebo mice fed a HFHC diet (FIGS. 2C and 2G). Next, a detailed study of levels of various sphingolipids was conducted using mass spectrometry. Treatment with 10 mg/kg non-encapsulated D-PDMP, 1 mg/kg D-PDMP encapsulated in biopolymer, or 10 mg/kg D-PDMP encapsulated in biopolymer did not reduce levels of glucosylceramide (GlcCer); instead, such treatments increased GlcCer levels in the liver (FIG. 2D). In contrast, 10 mg/kg non-encapsulated D-PDMP, 1 mg/kg D-PDMP encapsulated in biopolymer, and 10 mg/kg D-PDMP encapsulated in biopolymer, in a descending manner, reduced liver levels of lactosylceramide (LacCer; FIG. 2E). Levels of ceramide were not significantly different in livers of the various groups of mice (FIG. 2H). Similarly, levels of sphingosine-1-phosphate (SIP) remained unchanged in livers of placebo mice or mice treated with 10 mg/kg non-encapsulated D-PDMP (FIG. 2I). Biopolymer encapsulated D-PDMP appeared to affect lactosylceramide synthase, resulting in a marked concentration-dependent decrease in lactosylceramide levels.

### Example 3: Biopolymer Encapsulated D-PDMP Interfered with Cardiac Hypertrophy in apoE −/− Mice Fed a HFHC Diet

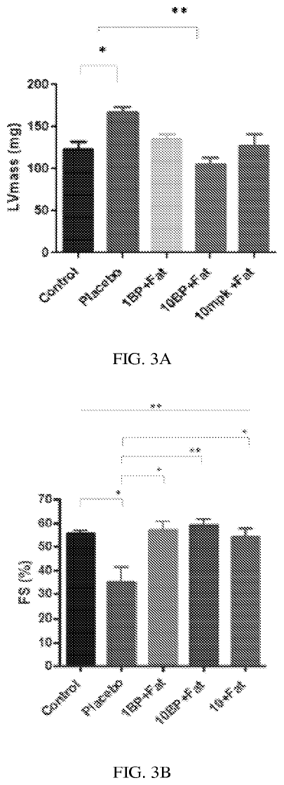

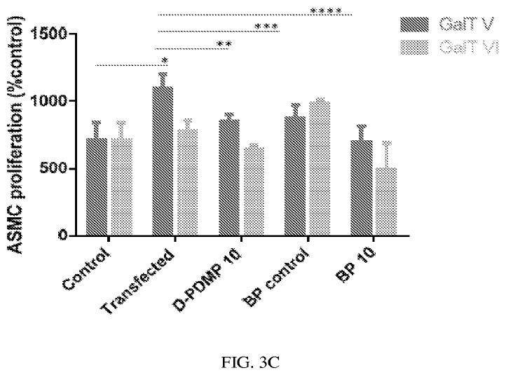

apoE −/− mice fed a HFHC diet showed marked atherosclerosis and increases in blood levels of lipids and lipoproteins, vascular stiffness, and increases in aortic media intima thickening. Collectively, these changes increased a heart's left ventricular mass (LVmass; FIG. 3A) and decreased fractional shortening (FS; FIG. 3B). apoE −/− mice fed a HFHC diet and treated with 1 mg/kg D-PDMP encapsulated in biopolymer from week 24 to week 36, demonstrated a markedly decreased left ventricular mass (FIG. 3A) and also increased fractional shortening (FIG. 3B). Such changes were comparable to mice treated with 10 mg/kg D-PDMP encapsulated in biopolymer or 10 mg/kg non-encapsulated D-PDMP. Together, treatments with biopolymer-encapsulated D-PDMP prevented onset of cardiac hypertrophy.

Additionally, D-PDMP encapsulated in biopolymer was more effective in mitigating airway smooth muscle cell (ASMC) proliferation induced by lactosylceramide (LCS) overexpression (FIG. 3C). ASMC were transfected with control, β-1,4-galactosyltransferase V (GalT V), or β-1,4-galactosyltransferase VI (GalT VI) expression constructs. Twelve hours after transfection, cells were treated with non-encapsulated D-PDMP and D-PDMP encapsulated in biopolymer. Cells were simultaneously treated with 5 μCi/ml of H³-Thymidine.

### Example 4: Biopolymer Encapsulated D-PDMP Markedly Altered Expression of Genes Implicated in Cholesterol Homeostasis and Cardiac Hypertrophy

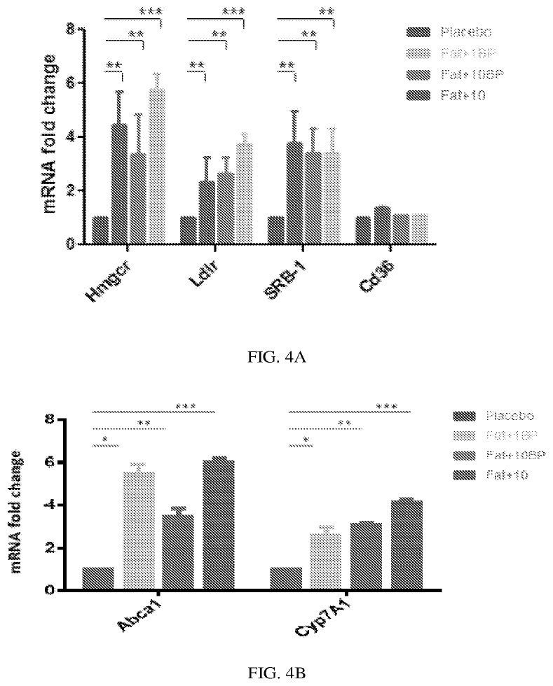

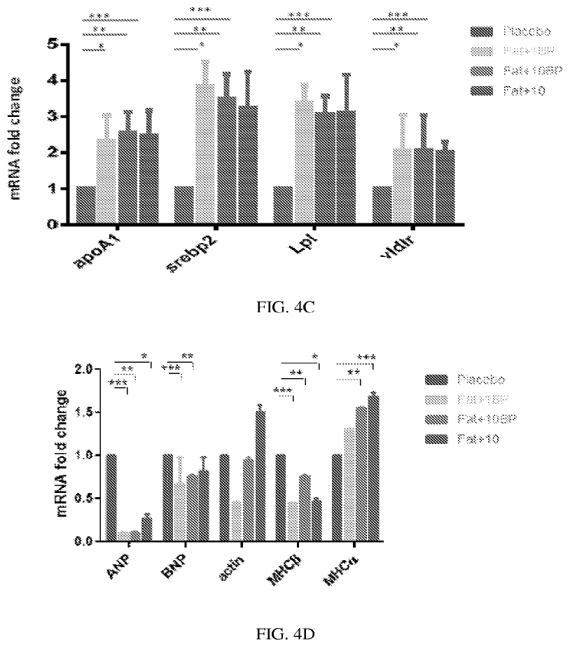

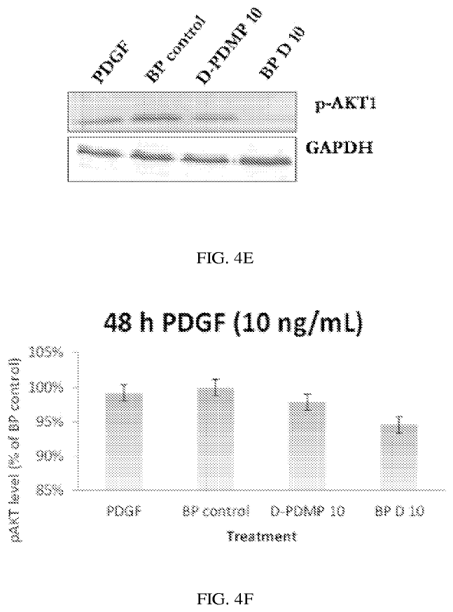

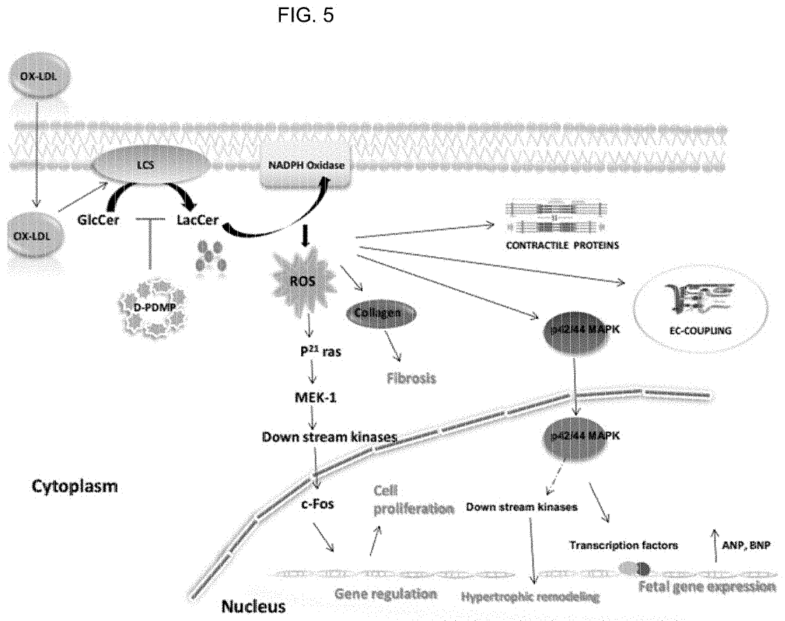

RT-PCR assays revealed that feeding a western diet to apoE−/− mice decreased the liver mRNA level of LDL receptors (Ldlr), scavenger receptor class b type 1 (SRB1), and HMG-CoA reductase (HMGcr), the key enzyme involved in the regulation of cholesterol biosynthesis (FIG. 4A). In contrast, apoE −/− mice fed the HFHC diet treated with 1 mg/kg D-PDMP encapsulated in biopolymer or with 10 mg/kg D-PDMP encapsulated in biopolymer have increased LDLr mRNA levels and HMG-Cr mRNA levels. Encapsulated D-PDMP treatments also raised mRNA levels of scavenger receptor class B (SRB-1; FIG. 4A) and Sterol regulatory element binding transcription factor (Srebp2; FIG. 4C) in the liver. Mice fed the HFHC diet and treated with 10 mg/kg non-encapsulated D-PDMP had increased mRNA levels of LDLr, HMGCr, and SRB-1 similar to the increased levels of mice treated with 1 mg/kg D-PDMP encapsulated in biopolymer. These data suggested that 1 mg/kg D-PDMP encapsulated in biopolymer was ten times as effective as 10 mg/kg of non-encapsulated D-PDMP in interfering with atherosclerosis via increasing expression of genes relevant to cholesterol metabolism. In contrast, the mRNA level of CD36 antigen was unchanged among the various experimental groups (FIG. 4A).

ATP-binding cassette sub-family ABCA1 (Abca1) is known to regulate egress of cholesterol from peripheral tissues back to the liver. Consistent with this role, liver mRNA levels of this gene increased approximately six-fold in mice fed the HFHC diet and treated with 1 mg/kg D-PDMP encapsulated in biopolymer (FIG. 4B). Cholesterol-7-alpha-hydroxylase (Cyp7A1) is required for the conversion of cholesterol to bile acids; liver mRNA levels of this gene increasedapproximately four-fold in mice fed the HFHC diet and treated with 1 mg/kg D-PDMP encapsulated in biopolymer when compared to mice fed the HFHC diet alone.

Apolipoprotein A-1 (apoA-1) is the major protein component in high density lipoproteins and studies have suggested that apoA-1 has an amino acid motif which binds to cholesterol and carries it out of cells, transporting it to the liver, where it is converted to bile acids. Liver mRNA levels of this gene increased two- to five-fold in apoE −/− mice fed the HFHC diet and treated with 1 mg/kg D-PDMP encapsulated in biopolymer (FIG. 4C). Without wishing to be bound by theory, lipoprotein lipase (Lpl) serves as a conduit to bind to circulating triglyceride rich lipoproteins (LPL) such as very low density lipoprotein (VLDL) and VLDL receptor (VLDLr), thereby facilitating delivery of VLDL to the liver and its subsequent catabolism. Liver mRNA levels of LPL and VLDLr increased two- to three-fold in apoE −/− mice fed the HFHC diet and treated with 1 mg/kg D-PDMP encapsulated in biopolymer (FIG. 4C). Apparently, feeding a HFHC diet to apoE −/− mice adversely down regulated expression of key genes involved in cholesterol, triglyceride, and bile acid metabolism and genes implicated in cholesterol efflux. Encapsulating a glycosphingolipid glycosyltransferase inhibitor within a biopolymer markedly interfered with atherosclerosis via increasing expression of genes critical to metabolism of lipids and lipoproteins.

Atrial natriuretic peptide (ANP), brain natriuretic peptide (BNP), and myosin heavy chain beta (MHC-β) are biomarkers of cardiac hypertrophy. The mice fed an HFHC diet exhibited markedly increased mRNA levels of ANP, BNP and MHC-β (FIG. 4D). Heat map mRNA levels of ANP, BNP and MHC-β decreased two- to ten-fold in apoE −/− mice fed the HFHC diet and treated with 1 mg/kg D-PDMP encapsulated in biopolymer (FIG. 4D); this treatment was significantly superior to treatment with 10 mg/kg of non-encapsulated D-PDMP. Biopolymer encapsulation of D-PDMP not only interfered with atherosclerosis in apoE −/− mice fed a HFHC western diet but was also cardioprotective.

Treatments with biopolymer encapsulated D-PDMP inhibited pAKT expression when compared to treatments with non-encapsulated D-PDMP (FIGS. 4E and 4F).

Without wishing to be bound by theory, atherosclerosis involves a highly charged oxidative stress environment. Herein, large increases in LDL, and low anti-oxidant status in the sub-endothelial space, may well lead to its conversion to oxidized LDL. As a defense mechanism, such oxidized LDL is taken up by macrophages via various scavenger receptors, and subsequently it may form fatty streaks and atherosclerotic plaques. Blood levels of glycosphingolipids rise and fall in tandem with the increase in LDL cholesterol as demonstrated with familial hypercholesterolemic patients who underwent plasma exchange therapy (Chatterjee PNAS 1986). However, such LDL particles decrease lactosylceramide synthase (LCS) activity. Rather, it is the oxidized LDL which stimulate the activity of LCS to generate lactosylceramide. Also pro-inflammatory cytokines such as, e.g., TNF-A and growth factors (e.g. VEGF, FGF, EGF, PDGF) released from activated vascular cells, platelets and macrophages can activate LCS to collectively raise the level of LacCer. This pool of endogenously synthesized LacCer partakes in ROS generation and downstream activation of p44MAPK to induce several genes.

Glycosphingolipids play an important role in atherosclerosis. For example, it has been documented that lactosylceramide in particular can activate endothelial cells to express ICAM-1, PECAM-1 and monocytes/neutrophils to express CD11b/Mac-1, which allows the adhesion and trans-endothelial migration of monocytes and neutrophils-the first step in inflammation and atherosclerosis. Additionally, LacCer serves as a bona fide mitogenic agent to induce arterial smooth muscle cell proliferation “a hallmark in the pathogenesis in atherosclerosis” (see e.g., Chatterjee BBRC 1991, ATVB 1998) and also induce angiogenesis. More importantly, LacCer can also induce ROS generation to stimulate smooth muscle cell proliferation (Bhunia J B C 1977) and cardiac hypertrophy. Other studies have shown that LacCer inhibits the expression of an ABC cassette-1 gene/protein responsible for the reverse transport of cholesterol from peripheral tissues back to the liver. On the other hand, in monocytes and neutrophils, LacCer activates phospholipase A2, which produces arachidonic acid, a precursor for pro-inflammatory prostaglandins. Thus, increased levels of LacCer and associated lipids are important for atherosclerosis.

As described in detail above, mass spectrometry and Gamma scintigraphy using an X-SPECT-SPECT-CT scanning have been used to quantitatively compare the kinetics of release and bio-distribution of a glycosyltransferase inhibitor, D-PDMP, with and without biopolymer encapsulation in mice. Ultra sound imaging, MALDI-MS-MS, and other routine biomolecular methods have also been used to compare the efficacy of the native D-PDMP and biopolymer-encapsulated D-PDMP to interfere with atherosclerosis and cardiac hypertrophy in apoE−/− mice fed a western diet composed of high fat and cholesterol.

The results presented herein indicated that the encapsulation of D-PDMP within a biopolymer allowed rapid absorption from the gastrointestinal tract and increased residence time ˜48 hr in the body of the mice. In comparison, the residence time of the native D-PDMP was about an hour. Advantageously, the net gain of treatment with the biopolymer-encapsulated D-PDMP was at least a 10-fold increase in efficacy in ameliorating atherosclerosis and cardiac hypertrophy. This was demonstrated by a marked decrease in lipid load and increased lumen volume in the aorta. Thus, atherosclerosis was interfered at 1 mpk D-PDMP encapsulated within the biopolymer (at a greater efficacy than 10 mpk D-PDMP) due to a reduction in the levels of several glycosphingolipids and bulk lipids such as cholesterol and triglycerides. The level of LDL cholesterol was decreased due to the increased expression of several genes implicated in LDL catabolism (e.g., LDL receptor, SREBP2). The level of HDL was increased as the expression of its major constituent protein apoA-1 was increased. The blood levels of triglycerides were markedly decreased due to an increase in the expression of VLDL receptors and lipoprotein lipase. Previous studies have shown that VLDL is the major carrier of triglycerides and lipoprotein lipase can serve as a conduit by binding to triglyceride rich particles and binding to the VLDL receptor. Here, it was observed that increased expression of genes such as ABC-A-1, ABCG5, and ABCG8 responsible for the efflux of cholesterol from liver and intestine for excretion in treated mice compared to placebo. Also the expression of the gene Cyp7A1, responsible for the expression of an enzyme 7-hydroxylase which converts cholesterol to bile acid is increased in treated mice. Importantly, fractional shortening, which is an indicator of the contraction of heart as well as left ventricular mass (a marker of cardiac hypertrophy), was returned to normal levels in treated apoE−/− mice compared to mice fed a western diet.

The results herein show that it was possible to mitigate/ameliorate atherosclerosis by feeding mice as little as 1 mpk D-PDMP encapsulated within a biopolymer. In addition, the results herein show that feeding a western diet for 20 weeks markedly increased left ventricular hypertrophy and decreased fractional shortening, an index of the contraction of the heart, measured by Doppler. Also several biomarkers of hypertrophy such as the expression of atrial natriuretic factor and brain natriuretic factor gene expression were increased in the left ventricle in mice fed a western diet. In contrast, feeding 1 mpk of biopolymer encapsulated D-PDMP interfered with cardiac hypertrophy. This increase (at least 10-fold) in the efficacy of biopolymer encapsulated D-PDMP compared to native D-PDMP may be explained due to rapid absorption and increased residence time. In the recent past, D-PDMP has been widely used to study the role of glycosphingolipids, various phenotypes and animal models of human diseases in vitro and in vivo (Chatterjee, S. Plos One 2013, Circulation 2014). Therefore, the compositions and methods set forth herein, which encapsulate D-PDMP within a biopolymer, will accelerate research in this field and significantly reduce the cost of such studies. Moreover, since both polyethelene glycol and sebacic acid are FDA approved, the results herein may help facilitate human trials of biopolymer-encapsulated D-PDMP to interfere with atherosclerosis and cardiac hypertrophy in hyperlipidemic man in the very near future. Additionally, echocardiogram data has shown the presence of extensive calcification in the aorta in apoE−/− mice fed a western diet for 36 weeks. In addition to the above-described results, treatment with 1 mg/kg of Biopolymer encapsulated D-PDMP resulted in extensive aortic decalcification.

### Example 5: Biopolymer Encapsulated D-PDMP Ameliorates Hyperlipidemia and Atherosclerosis

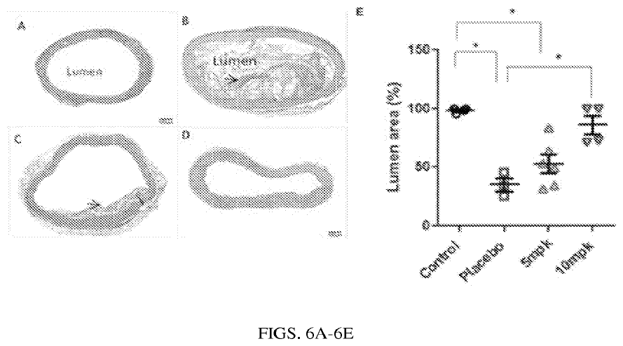

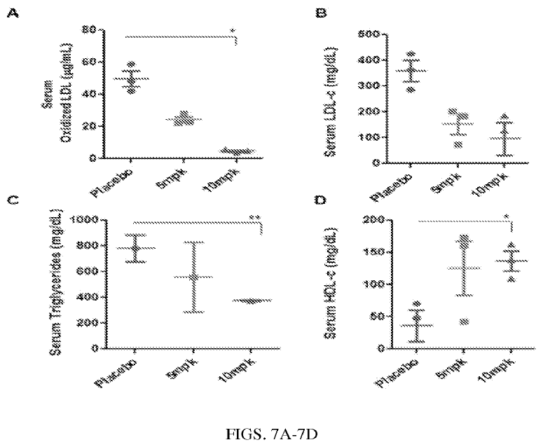

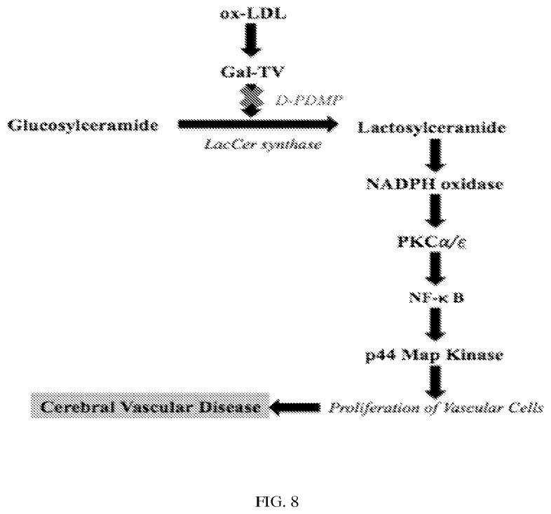

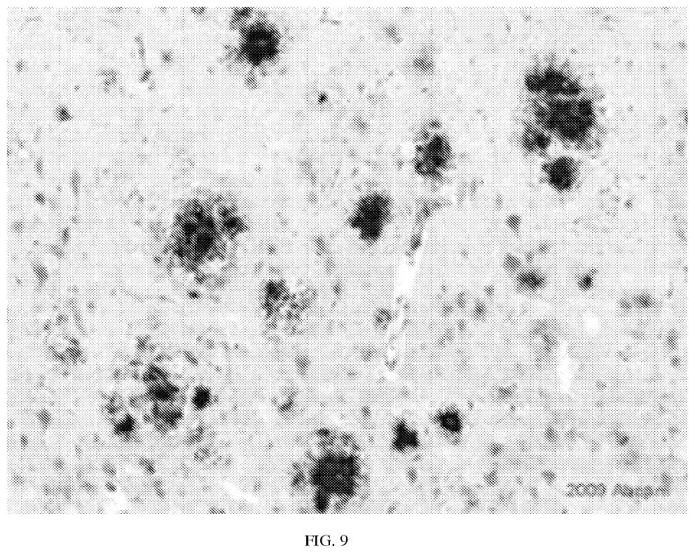

In vivo data generated in the well-accepted Apo E−/−mouse model of diet-induced hyperlipidemia and atherosclerosis showed that biopolymer encapsulated D-PDMP ameliorates hyperlipidemia and atherosclerosis. As shown in FIGS. 6 and 7, biopolymer encapsulated D-PDMP interfered with atherosclerosis and reversed the aortic intima media thickening and deposition of Ca 2+(3) (see e.g., FIGS. 6 and 7): an effect never before achieved by any other cholesterol lowering drug on the market.

As shown in FIG. 6, BDP-encapsulated D-PDMP treatment ameliorates atherosclerotic plaque buildup and lumen volume in ApoE−/− mice fed a western diet. FIGS. 6A-6D show Masson Trichrome stained ascending aortic rings of ApoE−/− mouse. In FIG. 6A, control mice were fed regular mice chow. In FIGS. 6B-6D, mice were fed a high fat, high cholesterol (HFHC) diet consisting of 20% fat and 1.25 cholesterol plus vehicle (Placebo; FIG. 6B), HFHC+5 mg/kg D-PDMP (FIG. 6C), and HFHC+10 mg/kg D-PDMP (FIG. 6D). FIG. 6E shows that the lumen area is significantly reduced due to increased plaque accumulation in placebo mice aorta. In other words, treatment significantly reduced medial thickening, elastin fibers, and plaque accumulation in a dose-dependent manner, which was confirmed using a nonparametric one-way ANOVA using the Kruskal-Wallis test and Dunn's multiple comparison post-test were performed. * p≤0.05, ** p≤0.01, *** p≤0.001; n=3-5.

As shown in FIG. 7, treatment also markedly reduced LDL cholesterol, oxidized LDL cholesterol, triglycerides and raised the levels of HDL cholesterol significantly. Plasma levels of oxidized LDL, cholesterol, triglycerides, and HDL-c in ApoE−/− mice fed a high fat and high cholesterol diet with and without BDP-encapsulated D-PDMP were measured. FIG. 6 shows serum levels of oxLDL (FIG. 6A), LDLc (FIG. 6B), triglycerides (FIG. 6C), and HDLc (FIG. 6D) were determined using an immunohistochemical ELISA assay and LDLc triglycerides and HDLc concentrations were taken from microtiter readings following Wako kit assays.

The increase in HDL levels was significantly higher than that reported with the use of statins, which may facilitate the reverse transport of cholesterol from peripheral tissue back to the liver. In liver, such cholesterol was converted to bile acids due to the activation of the gene CyP7A1. Biopolymer encapsulated D-PDMP was tolerated by experimental animals very well, including mice and rabbits, even when given at 10 times the effective dose up to 6 months. In fact, there was a modest increase in body weight due to increased bone density. Additionally, adipocytes were made insulin resistant by chronic exposure to low levels of tumor necrosis factor (TNF). In contrast, treatment with D-PDMP reversed the TNF induced impairment in insulin signaling. These observations suggest additional benefits of treatment with D-PDMP by way of improved insulin signaling. In view of the foregoing, it is clear that biopolymer encapsulated D-PDMP may be used as a glycosphingolipid synthesis inhibitor that is effective in treating not only atherosclerosis, but also for preventing atherosclerosis in patients with Type 2 Diabetes Mellitus. It may also serve as an alternative drug for patients who are allergic to and/or cannot tolerate statins, as discussed above. This latter patient population is estimated at 10 million in the US alone.

It is contemplated within the scope of the invention that biopolymer encapsulated D-PDMP may be used to address multiple issues in atherosclerotic heart disease via, for example: increasing cholesterol efflux from the peripheral tissues to liver where it was efficiently converted to bile acid and excreted; reducing blood levels of LDL cholesterol; and/or increasing blood levels of HDL, unlike statins. Additionally, biopolymer encapsulated D-PDMP is 1000 times more efficacious than other glycosphingolipid lowering compounds. Importantly, glucose homeostasis was remarkably well regulated with biopolymer encapsulated D-PDMP, but not with other GSL lowering compounds.

Additionally, studies using an in vitro model of wounding in cultured human arterial cell monolayer revealed that treatment with D-PDMP facilitated healing. Thus, D-PDMP may well facilitate wound healing in diabetic mice and man. D-PDMP may not only improve the diabetic condition of the patient, but also simultaneously either prevent the development of atherosclerosis or treat any atherosclerosis that the patient already has. Additionally, to date no toxicity to D-PDMP has been observed in preclinical studies in mice and rabbits. In the event that a lack of efficacy at the desired dose of biopolymer encapsulated D-PDMP is observed, the dose may be raised, or the drug may be chemically modified.

### Example 6: Administration of a Glycosyltransferase Inhibitor was Observed to Treat or Prevent Cerebrovascular Disease

The role of cholesterol and glycosphingolipid glycosyltransferase in regulating amyloid-β levels, a well-established biomarker in Alzheimer's disease (AD), was investigated with the goal of experimentally identifying whether a glycosyltransferase inhibitor (here, D-PDMP) could treat or prevent a cerebrovascular disease of the brain-specifically, cerebral atherosclerosis with noted impact upon Alzheimer's disease/amyloid-β plaque accumulation. It was found that feeding a high fat and cholesterol diet to apoE−/− mice, a well-established mouse model of atherosclerosis, increased the levels of oxidized low density lipoproteins (ox-LDL) and lactosylceramide synthase (GalT-V) in serum and brain tissue. In contrast, treatment with a glycosyltransferase inhibitor (D-PDMP) dose-dependently decreased the levels of ox-LDL, GalT-V and amyloid-β levels in serum and in brain tissue. Thus, lactosylceramide synthase was confirmed as a target for amelioration of the pathophysiology of cerebrovascular diseases, specifically Alzheimer's disease.

Atherosclerosis can affect all parts of the vascular system, including the brain and the blood vessels that provide the brain with proper glucose levels. An unhealthy diet has been previously shown to correlate with primary hypertension, which in turn increases fat and cholesterol content in the artery wall. This accumulation restricts blood flow to the brain, thereby inducing cerebrovascular diseases/events such as strokes. Only a few studies have previously addressed the relationship between these two diseases. While it is often fatal when atherosclerosis occurs in the brain, the molecular link between the disease and its causes has not been fully established.

Effective drugs for such diseases are hard to design. As also described elsewhere herein, D-PDMP works by inhibiting lactosylceramide synthesis (refer to FIG. 8), which causes fatty streaks to accumulate in blood vessels. Studies described herein and recently published have demonstrated that cholesterol levels can be reduced in mice given a high-fat diet and the D-PDMP drug, with little or no side effects (Chatterjee et al. Circulation 2014; 129:23). Other biomarkers of atherosclerosis were measured to see if the drug affected the whole mechanism of atherosclerotic pathogenesis. These proteins are important because ox-LDL activates GalT-V and generates lactosylceramide during atherosclerotic pathogenesis. However, cholesterol also plays a significant role in regulating amyloid-β levels. With high levels of cholesterol, amyloid-β is not broken down as usual. Instead, amyloid-β forms into large plaques often found in brain tissues of patients with Alzheimer's disease (refer to FIG. 9; Leduc V, Jasmin-Belanger S, Poirier J. ApoE and cholesterol homeostatis in Alzheimer's disease). Since D-PDMP reduces levels of cholesterol and mitigates the disease's effects, treatment with D-PDMP was examined for the ability to yield a similar result with amyloid-β, thereby providing an alternative to standard Alzheimer's therapy.

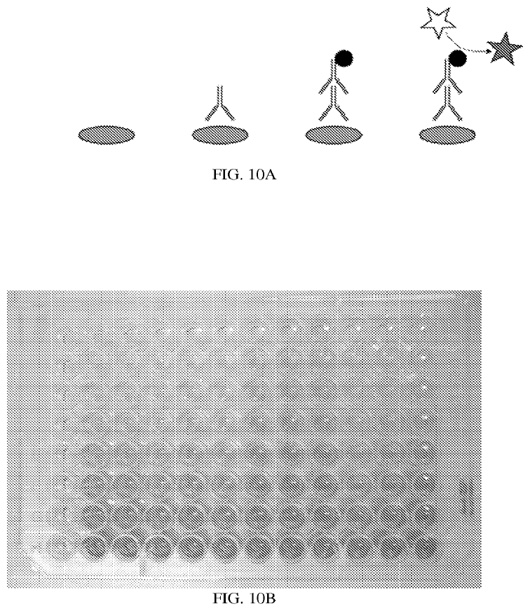

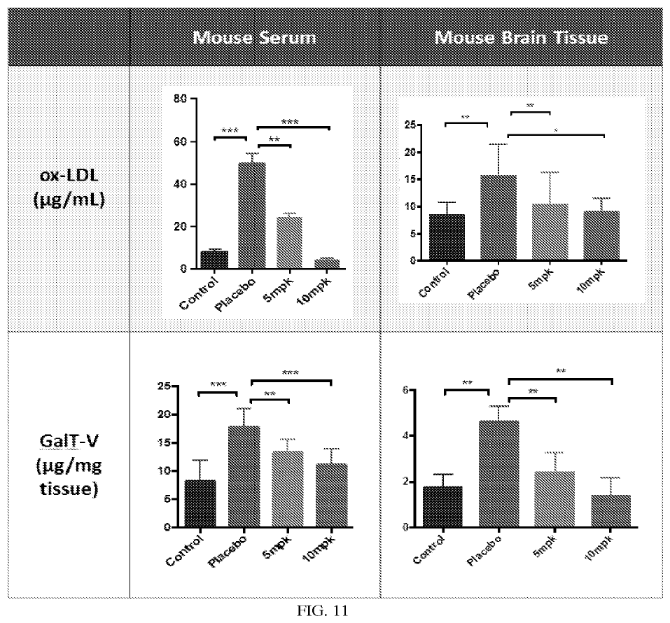

Testing of the impact of D-PDMP in a mouse AD model and quantification of biomarkers in such studies was performed in the following manner. Apolipoprotein E−/− mice were used because they exhibited exaggerated effects of atherosclerosis. These mice were fed either a normal diet (control) or a high-fat high-cholesterol (HFHC) diet. While a subset of the mice fed the high-fat diet was retained as a placebo group, others were treated with 5 mg/kg (mpk) or 10 mpk of the D-PDMP drug. After euthanizing these animals, tissue and blood samples were stored for further biochemical analysis. For the quantification of the biomarkers, enzyme-linked absorbent assay (ELISA) was used. For the measurement of ox-LDL, mouse serum was coated onto the bottom of a well. Then, a primary antibody bound specifically to the ox-LDL found in the serum. A secondary antibody was administered to that attached specifically to the primary antibody. This secondary antibody presented an attached reporter, allowing amounts of ox-LDL could be quantified. A substrate was bound to the reporter and fluoresced. In a sample with a higher concentration of ox-LDL, greater fluorescence was observed (refer to FIG. 10A). ELISA was also employed for quantifying levels of GalT-V and amyloid-β, using different target-specific antibodies.

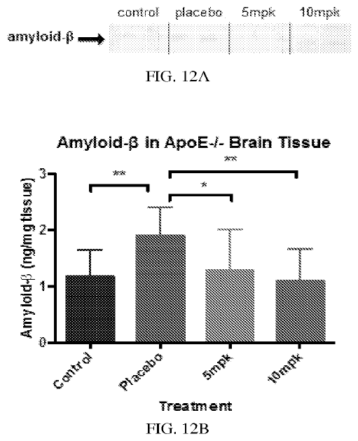

As shown in FIG. 11, administration of D-PDMP decreased atherosclerotic biomarkers in treated AD model mice, in a dose-dependent manner. While the placebo group (mice fed HFHC diet) exhibited higher levels of both ox-LDL and GalT-V than normal mice, those treated with the drug (5 mpk and 10 mpk, administered by injection and in this Example, noted as not biopolymer-encapsulated) had decreased levels of the biomarkers, and dose-dependence was observed.

D-PDMP administration was also observed to have decreased amyloid-β in treated ApoE−/− mice. Western blot revealed only small amounts of amyloid-β in brain tissue samples (FIG. 12A); and while amyloid-β levels were observed to have increased in brain tissue in mice fed HFHC diet, D-PDMP administration resulted in reduction of amyloid-β in brain tissue samples, also in an apparently dose-dependent manner (FIG. 12B).

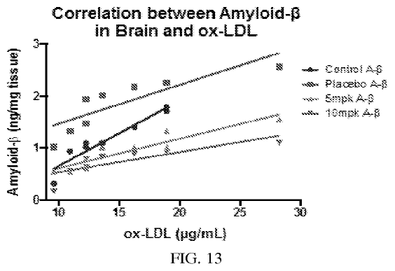

Thus, it was identified that ox-LDL cholesterol regulated the levels of both GalT-V and amyloid-β; and treatment with D-PDMP markedly reduced the levels of atherosclerosis and Alzheimer's disease biomarkers in treated ApoE −/− model mice. In addition, reduction of ox-LDL and amyloid-β were observed to be linearly correlated, with D-PDMP administration reducing the levels of both biomarkers (FIG. 13). Indeed, positive correlation was detected in all mice between increasing levels of ox-LDL and increasing levels of amyloid-β, further indicating the molecular/physiologic relationship between the former atherosclerosis biomarker and the latter AD marker.

### Example 7: Prophylaxis of Atherosclerosis and Diabetes Using the db/db Mouse Model

High levels of blood LDL cholesterol and triglycerides in addition to low levels of HDL cholesterol accompany type II diabetes. The mouse model mice employed herein were also unable to handle glucose properly due to resistance to insulin. It is believed that biopolymer encapsulated D-PDMP can not only ameliorate the pathology due to hyperlipidemia but also help facilitate glucose homeostasis in these diabetic mice. To test this, a mouse (N=10 in each group) model of type II diabetes ((db/db) −12 week old) are fed a western diet (20% fat and 1.25% cholesterol, meaning a high fat and cholesterol rich diet, as compared to a placebo) and given biopolymer encapsulated D-PDMP (1 mpk and 10 mpk) daily by oral gavage from week 12-30. The control group of mice are fed vehicle (5% Tween 80 in PBS) by oral gavage only. Male db/db mice 12 weeks old are subjected to ultrasound imaging to establish baseline aortic intima thickening and pulse wave velocity. Mice (N=5) are euthanized, blood is collected and serum are prepared to measure various lipids and lipoproteins, glucose, glucagon, insulin and other parameters. Tissues are harvested and portions are fresh frozen for lipid analysis by tandem MS/MS method, gene expression assays using quantitative RT PCR. These include genes implicated in lipid and lipoprotein metabolism, for example: the PPAR's, insulin receptor, GSL glycosyltransferases, etc. Some tissue sections are fixed in formalin and subject to Masson's Trichrome staining (to quantify collagen deposition in the arterial wall, an index of fibrosis) and immunostaining using various antibodies to show the deposition of alpha smooth muscle cell actin, macrophages, etc. Mice are subjected to these studies again at the end of treatment (30 weeks). This experiment uses 60 mice. Based on previous experience with apoE−/− mice study with BPD above, statistically significant increases in aortic lumen volume (2 fold) and HDL levels (3 fold) and reductions in LDL and triglyceride levels (2-folds) in the treated group compared to placebo by 30 weeks indicates a very successful therapeutic outcome for biopolymer encapsulated D-PDMP. It is also likely that biopolymer encapsulated D-PDMP is identified to improve glucose levels, as can be measured by a decrease to within the normal range (100 mg/dL).

### Example 8: Treatment of Atherosclerosis and Diabetes Using the Db/Db Mouse Model

Diabetic mice (N=10 in each group) are fed a diet w/o the western diet from 12 weeks of age till 20 weeks of age. Next, all diabetic male mice (20 weeks of age) are subjected to ultrasound imaging. A group of these mice N=5 are euthanized, blood and tissues harvested and subjected to various immunohistochemical, biochemical, and molecular studies above. The rest of the mice are divided into the following groups: Group A continues feeding on the western diet, Group B continues feeding on the western diet and is given D-PDMP as a control, and Groups C, D, and E continue feeding on the western diet and are given 0.1 mpk, 1 mpk, and 10 mpk, respectively, of biopolymer encapsulated D-PDMP daily by oral gavage. At 24 weeks and 30 weeks of age, ultrasound imaging is conducted and followed by blood and tissue harvests and measurements of various parameters above. This experiment uses 150 mice. A statistically significant increase in aortic lumen volume and HDL levels and reductions in LDL and triglyceride levels by week 30 as well as improved glucose homeostasis as measured by a decrease of fasting plasma glucose from 200 mg/dl in the placebo group to 100 mg/dL in the treated group is considered a strongly therapeutic outcome.

The above experiments were performed using the following Methods and Materials

### Methods and Materials

The polyethylene glycol-sebacic acid (PEG-SA) copolymer was prepared as previously described (Fu J, et al. Biomaterials. 2002; 23: 4425-4433). Microparticles of D-PDMP encapsulated by the PEG-SA copolymer were prepared by modifying the single emulsion solvent evaporation method. For scintigraphic tracking of the biopolymer, the PEG polymer was radio-iodinated with 45 mCi (810 kBq) of [¹²⁵I]NaI. The radiolabeled PEG was then incorporated into the PEG-SA biopolymer. C57BL/6 adult female mice were given 45 mCi (810 kBq) each of the [¹²⁵I] drug-loaded biopolymer orally by gavage. The biopolymer movement in vivo was measured by γ-scintigraphy using an X-SPECT SPECT-CT scanner (Gamma Medica Ideas, North Ridge, Calif., USA). Apolipoprotein E deficient (apoE−/−) mice aged 12 weeks were fed a high fat, high cholesterol (20% fat, 1.25% cholesterol; HFHC) diet until 20 weeks old. At this point, the biopolymer alone, 1 mg/kg (1 mpk) or 10 mpk D-PDMP with or without PEG-SA encapsulation was administered for an additional 16 weeks. Ultrasound imaging and histopathology were performed as previously described (Habashi J P, et al. Circulation. 2009; 120: S963-S963 and Olson L E, et al. Cancer Res. 2003; 63: 6602-6606). At 36 weeks, serum and tissues were gathered and flash-frozen for biochemical studies via necropsy. Oxidized LDL was measured in serum using an established ELISA protocol (Horkko S, et al. The Journal of clinical investigation. 1999; 103: 117-128). Glycosphingolipids, cholesterol, and triglycerides were measured by MALDI-MSMS (5800 TOF/TOF, AB SCIEX, Framingham, Mass., USA).

Preparation of Biopolymer encapsulated D-PDMP

PEG-SA Co-polymer was prepared following the published literature procedure by Eu and coworkers. Briefly, sebacic acid prepolymer was made by refluxing sebacic acid (SA) in acetic anhydride followed by drying under high vacuum (evaporation), crystallized from dry toluene, washed with 1:1 anhydrous ethyl ether-petroleum ether and finally air dried. PEG prepolymer was made by refluxing of polyoxyethylene dicarboxylic acid in acetic anhydride, volatile solvents were removed under vacuum. The solid mass was extracted with anhydrous ether and air dried. The poly(PEG-SA) co-block polymer was then synthesized by the melt polycondensation method and characterized by proton NMR. Note that this copolymer has been extensively characterized for the composition and structural identity (Aich U, et al. Glycoconjugate journal. 2010; 27: 445-459).

Encapsulation of D-PDMP in poly(PEG-SA) (to prepare polymer-encapsulated drug subsequently referred to as BPD) followed by the melt polycondensation method described above for SA and PEG prepolymers but with the inclusion of D-PDMP at starting ratios of poly(PEG-SA) to D-PDMP of 70:30 by weight. Subsequently, microparticles were prepared using a single emulsion solvent evaporation method.⁵ Briefly, D-PDMP and PEG-SA were dissolved in chloroform (50 mg/mL) and emulsified into a 1.0% w/w poly(vinyl alcohol) aqueous solution under sonication condition keeping the temperature below 25° C. Particles were hardened by allowing chloroform to evaporate at room temperature while stirring for 12 h. Particles were collected and washed three times with double distilled water via centrifugation at 2,600×g (30 min) and lyophilized for 48 h before it was ready to use.

Preparation of [¹²⁵ I]-BP-D-PDMP and imaging and metabolic experiments.

Radiolabeling the biopolymer. 20 mg of L-tyrosine (0.14 mmol) was introduced to 45 mCi (810 kBq) of [¹²⁵I]NaI in 100 mL of PBS in a glass vial containing plated Iodogen (Pierce, Rockford Ill. USA). The radioiodinated reaction proceeded at room temperature for 12 minutes before withdrawing the supernatant. The supernatant was then added to 100 mg of O,O′bis[2-(N-succinimidal-succinylamino)ethyl]polyethylene glycol (MW 3,000, Aldrich, St. Louis Mo. USA) and this mixture was allowed to sit at room temperature for 1 hour. After 1 hour, the reaction mixture was then loaded onto a PBS-conditioned G25 Sephadex size-exclusion column (Pierce, Rockford Ill. USA) to remove any unreacted iodide and free tyrosine. The absence of free radioiodine and tyrosine in the eluate was confirmed using ITLC (Gelman strips, Vernon Hills Ill. USA) in ACD buffer (Sigma-Aldrice, St. Louis Mo. USA). The labeled PEG was then incorporated into the PEG-SA biopolymer.

γ-scintigraphy of biopolymer movement. Drug-loaded biopolymer [45 mCi (810 kBq) of the ¹²⁵I-labeled samples] was orally administered by gavage to each of three C57bl/6 adult males. The mice were anesthetized using 2.5% isoflurane gas in oxygen delivered via tent. The mice were lined up side-by-side directly over a high-resolution parallel hole collimator in an X-SPECT SPECT-CT scanner (GammaMedica Ideas, North Ridge Calif. USA). Scans consisted of several 10 min acquisitions through two hours post-tracer administration with a CT scan followed by additional acquisitions as indicated with accompanying CT scans (512 slice, 50 keV beam). The data were reconstructed using the manufacturer's software and co-registered using AMIDE (see e.g., (www)sourceforge.net). All scintigraphy images are displayed to scale with each other.

### Animals and Treatments

Apoliporotein E-deficient (ApoE (−/−), male mice aged. 12 weeks (Jackson Labs, Bar Harbor, Me.) were used. At the age of 12 weeks, the Apo E (−/−) mice were started on a high fat and high cholesterol diet (HFHC) of 4.5 kcal/g, 20% fat, and 1.25% cholesterol (D12108C, Research Diet Inc., New Brunswick, N.J.) until 20 weeks of age. A control group on normal diet consisting only of chow food was used for comparison.

At 20 weeks, mice on HFHC were started on treatment with D-PDMP 1 mpk, 10 mpk with and without biopolymer encapsulation respectively and compared to control fed only chow diet and placebo fed HFHC+Vehicle (100 μL of 5% Tween 80 in PBS) for another 16 weeks. Sample size of n=5-6 subjects per group was designated.

Treatments were delivered daily by gavage. Food was rationed once a week to estimate the weekly growth rate and food intake. Physiological studies and tissue harvest were performed around 12 and 36 weeks for molecular and histo-pathological studies. The 20 week time point was designed for the start of treatment according to previous unpublished studies showing a significant of plaque accumulation, significant increase in ascending aortic intimae media thickness IMT_AsAo and vascular stiffness using histopathology, ultrasound and pulse wave velocity (PWV) respectively.

Additional mice (n=3) were given the biopolymer-encapsulated drug via gavage for the same indicated times. Mice were necropsied at the time points shown. Tissues were homogenized (chloroform-methanol 2:1, v/v) and lipids extracted from the stomach, duodenum, liver, kidney and serum. The tissues were then analyzed by mass spectrometry, with the presence and abundance of the drug determined by MS/MS.

### Ultrasound Imaging

Vascular ultrasound was performed in conscious mice using the 2100 Visualsonic ultrasound imaging system equipped with MS400: 38 MHz MicroScan transducer (Toronto, Ontario, Canada) as previously described (Habashi J P, et al. Circulation. 2009; 120: S963-S963 and Olson L E, et al. Cancer research. 2003; 63: 6602-6606).

The aorta was first viewed in 2 dimensional (2D) mode and the parasternal long axes view. The ascending aorta external and internal dimensions and intimae media thickness were measured at end systolic phase and in blinded fashion so that the genotype and treatments were not revealed.

The intima media thickness (IMT) was measured from the ascending aortic wall and derived according to the following equation:

IMT (AsAo) (mm)=External Ascending aorta diameter (mm)—Internal Ascending aortic diameter (mm), where AsAo refers to ascending aorta.

All measurements were performed according to the guidelines set by the American Society of Echocardiography. For each mouse, three to five values for each measurement were obtained and averaged for evaluation

### Histopathology

Masson trichrome and Verhoeff-van Gieson staining of the ascending aorta was used to quantify fibrosis, wall thickening and assess for the elastin fibers structure and morphology, plaque, and Ca² accumulation, 16 weeks after treatment and 36 weeks HFHC diet and aging time. Bar=100 μm for aortic diameter and 50 μm aortic segment. 50 μm Nikon 801 Eclipse equipped with Nikon DS-Ell camera and NIS-Elements software (Nikon, Japan) were used for image analysis.

### Measurement of Oxidized LDL Levels

The serum level of oxidized LDL (oxLDL) was measured using an ELISA assay and monoclonal antibody⁸ against human oxLDL according to instructions given by the supplier (Avanti Polar Lipids, Alabaster, Ala.).

### Measurement of Glycosphingolipids, Cholesterol and Triglycerides

A MALDI-TOF/TOF (AB SCIEX TOF/TOF 5800, Applied Biosystems, Framingham, Mass.) was used in this study for both MS and MS/MS analyses of glycosphingolipids, cholesterol, and triglycerides. Extracted lipids were reconstituted in 100 μL acetonitrile-methanol (9:1, v/v) and approximately 0.5 μL was spotted on an AB SCIEX Opti-TOF MALDI plate. Sample spots were overlaid with 0.5 μL of the MALDI matrix 2,5-dihydroxybenzoic acid (DHB) in an acetonitrile-methanol solution (5 mg/mL DHB in 9:1 ACN-MeOH, v/v). A 355 nm laser at a repetition rate of 200 Hz was employed for ionization. For MS analysis, mass spectra were the sum of 4000 laser shots and acquired in reflector positive mode. For MS/MS analysis, mass spectra were the sum of 1000 laser shots with a collision energy of 2 keV, and pressurized air was utilized as the collision gas to induce fragmentation. The following standards were used to calibrate the mass spectrometer: polypeptide hormones (ACTH I-III, Sigma-Aldrich, St. Louis, Mo.) and a peptide (Fibrinopeptide B, human, Sigma-Aldrich, St. Louis, Mo.).

The serum level of oxidized LDL (oxLDL) was measured using an ELISA assay and monoclonal antibody_ENREF_77 against human oxLDL according to instructions given by the supplier (Avanti Polar Lipids, Alabaster, Ala.).

### Quantitative Real-Time PCR

An approximately 50-mg piece of liver tissue was homogenized from each subject and total RNA was isolated using TRIzol reagent according to the manufacturer's instructions (Invitrogen). Two micrograms of RNA was reverse-transcribed with SuperScript II (Invitrogen, USA) using random primers. Real-time PCR was performed using SYBR Green PCR Master Mix (Applied Biosystems, USA) in an Applied Biosystems Step one Real time PCR system with the following thermal cycling conditions: 10 min at 95° C., followed by 40 cycles of 95° C. for 15 s and 60° C. for 1 min for denaturation, annealing and elongation. Relative mRNA levels were calculated by the method of 2^{-DDct}. Data were normalized to GAPDH mRNA level. To determine the specificity of amplification, melting curve analysis was applied to all final PCR products. All samples were performed in triplicate. Primers were synthesized by Integrated DNA Technologies (Coralville, USA). Expression suite software (Applied Biosystems) was used to analyze the data.

### REFERENCES

1. Chatterjee S B, Dey S, Shi W Y, Thomas K, Hutchins G M. Accumulation of glycosphingolipids in human atherosclerotic plaque and unaffected aorta tissues. Glycobiology. 1997; 7:57-65
2. Dawson G, Kruski A W, Scanu A M. Distribution of glycosphingolipids in the serum lipoproteins of normal human subjects and patients with hypo- and hyperlipidemias. J Lipid Res. 1976; 17:125-131
3. Chatterjee S, Sekerke C S, Kwiterovich P O, Jr. Alterations in cell surface glycosphingolipids and other lipid classes of fibroblasts in familial hypercholesterolemia. PNAS. 1976; 73:4339-4343
4. Meutermans W, Le G T, Becker B. Carbohydrates as scaffolds in drug discovery. ChemMedChem. 2006; 1:1164-1194
5. Fu J, Fiegel J, Krauland E, Hanes J. New polymeric carriers for controlled drug delivery following inhalation or injection. Biomaterials. 2002; 23:4425-4433
6. Habashi J P, Holm T_{M}, Doyle J J, Aziz H, Bedja D, Dietz H C. At2 signaling is a positive prognostic and therapeutic modifier of marfan syndrome: Lessons on the inequality of acei and arbs. Circulation. 2009; 120:S963-S963
7. Olson L E, Bedja D, Alvey S J, Cardounel A, Gabrielson K L, Reeves R H. Protection from doxorubicin-induced cardiac toxicity in mice with a null allele of carbonyl reductase 1. Cancer Res. 2003; 63:6602-6606
8. Horkko S, Bird D A, Miller E, Itabe H, Leitinger N, Subbanagounder G, Berliner J A, Friedman P, Dennis E A, Curtiss L K, Palinski W, Witztum J L. Monoclonal autoantibodies specific for oxidized phospholipids or oxidized phospholipid-protein adducts inhibit macrophage uptake of oxidized low-density lipoproteins. The Journal of clinical investigation. 1999; 103:117-128
9. Aich U, Meledeo M A, Sampathkumar S-G, Fu J, Jones M B, Weier C A, Chung S Y, Tang B C, Yang M, Hanes J, Yarema K J. Development of delivery methods for carbohydrate-based drugs: Controlled release of biologically-active short chain fatty acid-hexosamine analogs. Glycoconjugate journal. 2010; 27:445-459
10. Olson L E, Bedja D, Alvey S J, Cardounel A J, Gabrielson K L, Reeves R H. Protection from doxorubicin-induced cardiac toxicity in mice with a null allele of carbonyl reductase 1. Cancer research. 2003; 63:6602-6606
11. Kwiterovich P. The Johns Hopkins textbook of dyslipidemia. Philadelphia: Wolters Kluwer Health/Lippincott Williams & Wilkins; 2010.
12. Garner B, Priestman D A, Stocker R, Harvey D J, Butters T D, Platt F M. Increased glycosphingolipid levels in serum and aortae of apolipoprotein e gene knockout mice. J Lipid Res. 2002; 43:205-214.
13. Radin N S, Shayman J, Inokuchi J-I. Metabolic effects of inhibiting glucosylceramide synthesis with pdmp and other substances. Advances in lipid research. 1993; 26:183.
14. Boisvert W A, Black A S, Curtiss L K. Apoa1 reduces free cholesterol accumulation in atherosclerotic lesions of apoe-deficient mice transplanted with apoe-expressing macrophages. Arteriosclerosis, thrombosis, and vascular biology. 1999; 19:525-530.

### OTHER EMBODIMENTS

From the foregoing description, it will be apparent that variations and modifications may be made to the invention described herein to adopt it to various usages and conditions. Such embodiments are also within the scope of the following claims.

All citations to sequences, patents and publications in this specification are herein incorporated by reference to the same extent as if each independent patent and publication was specifically and individually indicated to be incorporated by reference.

We claim:

1. A composition for treating or reducing a symptom of atherosclerosis or cardiac hypertrophy comprising an effective amount of a biopolymer-encapsulated glycosphingolipid synthesis inhibitor.

2. The composition of claim 1, wherein the glycosphingolipid synthesis inhibitor is D-threo-1-phenyl-2-decanoylamino-3-morpholino-1-propanol (D-PDMP).

3. The composition of claim 2, wherein the D-PDMP is encapsulated in a polyethylene glycol-sebacic acid polymer.

4. The composition of any of the above claims, further comprising another therapeutic compound known to treat or reduce a symptom of heart disease generally and atherosclerosis or cardiac hypertrophy specifically.

5. The composition of any of the above claims, wherein the composition is configured for oral administration.

6. A method for treating or reducing a symptom of atherosclerosis or cardiac hypertrophy in a subject in need thereof comprising a step of providing a composition comprising an effective amount of a biopolymer-encapsulated glycosphingolipid synthesis inhibitor to the subject.

7. The method of claim 6, wherein the glycosphingolipid synthesis inhibitor is D-threo-1-phenyl-2-decanoylamino-3-morpholino-1-propanol (D-PDMP).

8. The method of claim 7, wherein the D-PDMP is encapsulated in a polyethylene glycol-sebacic acid polymer.

9. The method of any of claims 6-8, wherein the method comprises providing another therapeutic compound known to treat or reduce a symptom of heart disease generally and atherosclerosis or cardiac hypertrophy specifically.

10. The method of claim 7 or 8, wherein 0.1 mg to 100 mg of D-PDMP is provided per kg of subject bodyweight.

11. The method of claim 7 or 8, wherein 1 mg or 10 mg of D-PDMP is provided per kg of subject bodyweight.

12. The method of claim 7 or 8, wherein the method treats or reduces a symptom of atherosclerosis.

13. The method of claim 7 or 8, wherein the method treats or reduces a symptom of cardiac hypertrophy.

14. The method of any of claims 6-13, wherein the subject is a mammal.

15. The method of claim 14, wherein the mammal is a human.

16. The method of any of claims 6-15, wherein the composition is orally administered.

17. A composition for treating or reducing a symptom of diabetes comprising an effective amount of a biopolymer-encapsulated glycosphingolipid synthesis inhibitor.

18. The composition of claim 17, wherein the glycosphingolipid synthesis inhibitor is D-threo-1-phenyl-2-decanoylamino-3-morpholino-1-propanol (D-PDMP).

19. The composition of claim 18, wherein the D-PDMP is encapsulated in a polyethylene glycol-sebacic acid polymer.

20. The composition of any of claims 17-19, further comprising another therapeutic compound known to treat or reduce a symptom of diabetes.

21. The composition of any of claims 17-20, wherein the diabetes is Type 11 diabetes.

22. The composition of any of claims 17-21, wherein the symptom is selected from the group consisting of high levels of LDL cholesterol, low levels of HDL cholesterol, atherosclerosis, inability or delay of wound healing and blood glucose imbalance.

23. The composition of any of claims 17-22, wherein the composition is configured for oral administration.

24. A method for treating or reducing a symptom of diabetes in a subject in need thereof comprising a step of providing a composition comprising an effective amount of a biopolymer-encapsulated glycosphingolipid synthesis inhibitor to the subject.

25. The method of claim 24, wherein the glycosphingolipid synthesis inhibitor is D-threo-1-phenyl-2-decanoylamino-3-morpholino-1-propanol (D-PDMP).

26. The method of claim 25, wherein the D-PDMP is encapsulated in a polyethylene glycol-sebacic acid polymer.

27. The method of any of claims 24-26, wherein the method comprises providing another therapeutic compound known to treat or reduce a symptom of diabetes.

28. The method of any of claims 24-27, wherein the diabetes is Type II diabetes.

29. The method of any of claims 24-28, wherein the symptom is selected from the group consisting of high levels of LDL cholesterol, low levels of HDL cholesterol, atherosclerosis, inability or delay of wound healing and blood glucose imbalance.

30. The method of any of claims 24-29, wherein the composition is configured for oral administration.

31. The composition or method of any of claims 17-30, wherein 0.1 mg to 100 mg of D-PDMP is provided per kg of subject bodyweight.

32. The composition or method of any of claims 17-31, wherein 1 mg or 10 mg of D-PDMP is provided per kg of subject bodyweight.

33. The composition or method of any of claims 17-32, wherein the composition or method treats or reduces a symptom of atherosclerosis.

34. The composition or method of any of claims 17-33, wherein the subject is a mammal.

35. The composition or method of claim 34, wherein the mammal is a human.

36. The composition or method of any of claims 17-35, wherein the composition is orally administered.

37. The composition or method of any of claims 1-36, wherein the composition is a sustained or controlled release dosage formulation.

38. The composition or method of any of claim 6-16 or 37, wherein the cardiac hypertrophy is caused by a condition selected from the group consisting of cerebral atherosclerosis, obesity, metabolic syndrome, fibrosis of the lungs, fibrosis of the kidney, renal cancer, tuberculosis, Niemann-Pick Type C (NPC), Alzheimer's, epilepsy, and a metabolic disorder of glycosphingolipid metabolism.

39. The composition or method of claim 38, wherein the metabolic disorder of glycosphingolipid metabolism is selected from the group consisting of fibrosis of liver, inflammation (macrophage infiltration into the heart and coronary artery), Gaucher's disease (glucocerebrosidase deficiency) and Fabry's disease (ceramide trihexoside deficiency).

40. A method for treating or reducing a symptom of Alzheimer's disease (AD) in a subject in need thereof comprising a step of providing a composition comprising an effective amount of a glycosphingolipid synthesis inhibitor to the subject.

41. The method of claim 40, wherein the glycosphingolipid synthesis inhibitor is biopolymer-encapsulated.

42. The method of claim 40 or 41, wherein the glycosphingolipid synthesis inhibitor is D-threo-1-phenyl-2-decanoylamino-3-morpholino-1-propanol (D-PDMP).

43. The method of claim 41 or 42, wherein the glycosphingolipid synthesis inhibitor is encapsulated in a polyethylene glycol—sebacic acid polymer.

44. The method of any of claims 40-43, wherein amyloid-β levels are reduced and/or amyloid-β plaque levels and/or amyloid-β plaque formation is reduced or eliminated in the subject.
